(19) United States
(12) Patent Application Publication
(10) Pub. No.: US 2014/0148504 A1
(43) Pub. Date: May 29, 2014

(54) NOVEL USE OF FLAVONE-BASED COMPOUND

(71) Applicant: Industry-Academic Cooperation Foundation, Younsei University, Seoul (KR)

(72) Inventors: Jae Kwan Hwang, Gyeonggi-Do (KR); Jaekyung Kim, Seoul (KR)

(73) Assignee: Industry-Academic Cooperation Foundation, Yonsei University, Seoul (KR)

(21) Appl. No.: 13/946,609

(22) Filed: Jul. 19, 2013

Related U.S. Application Data
(63) Continuation of application No. PCT/KR2012/000601, filed on Jan. 25, 2012.

(30) Foreign Application Priority Data
Jan. 21, 2011 (KR) 10-2011-0006338

(51) Int. Cl. A61K 8/49; A61K 8/97; A61Q 19/08

(52) U.S. Cl. CPC A61K 8/498; A61Q 19/08; A61K 8/97
USPC 514/456; 549/403

(57) ABSTRACT

The present invention relates to a novel use of a flavone-based compound or a Kaempferia parviflora extract comprising the same. More particularly, the present invention relates to a composition for wrinkle improvement, anti-aging and skin elasticity enhancement or a composition for skin moisturization, which comprises a flavone-based compound or a Kaempferia parviflora extract comprising the same as an active ingredient. The composition of the present invention is very effective in inhibiting the activity of Collagenase-1 (MMP-1) and promoting the synthesis of collagen. Therefore, the composition of the present invention is useful for wrinkle improvement, anti-aging, skin elasticity enhancement, and skin moisturization via the inhibition of moisture loss from the skin.

### CROSS REFERENCE TO RELATED APPLICATION

The present application is a continuation of International Application No. PCT/KR2012/000601 filed on Jan. 25, 2012, which claims priority to Korean Application No. 10-2011-0006338 filed on Jan. 21, 2011, which applications are incorporated herein by reference.

### TECHNICAL FIELD

The present invention relates to a novel use of a flavone-based compound or a Kaempferia parviflora extract comprising the same. More particularly, the present invention relates to a composition for wrinkle improvement, anti-aging and skin elasticity enhancement or a composition for skin moisturization, which comprises a flavone-based compound or a Kaempferia parviflora extract comprising the same as an active ingredient.

### BACKGROUND ART

Aging is largely classified into natural aging or intrinsic aging, and extrinsic aging. Natural aging is caused by hereditary factors and is hard to control, whereas extrinsic aging is caused by environmental factors and can be controlled relatively easily. Accordingly, researches have continuously been conducted to prevent extrinsic aging. Especially, researches on the prevention of wrinkle formation caused by extrinsic photo-aging due to long-term exposure to UV have been drawing attentions (See Gilchrest B. A., J. Am. Acad. Dermatol., 1989:21:610-613). The photo-aging as a form of extrinsic skin aging is clinically characterized by rough and in-elastic skin, irregular pigmentation and increase of deep wrinkles.

External factors that affect the aging include wind, temperature, humidity, cigarette smoke, pollution, UV light, and the like. Especially, the aging caused by UV light is called photo-aging. Particularly, the photo-aging is deeply involved in wrinkle formation on the face and the head, which are cosmetically important areas. Therefore, basic researches on photo-aging and wrinkle formation on human skin or in animal model have been actively carried out for the development of anti-aging or anti-wrinkle cosmetics. With regard to photo-aging and wrinkle formation, changes in basic physiological metabolism such as synthesis and degradation of collagen, the main component of skin, have been reported (See Brenneisen et al., Ann N.Y. Acad. Sci., 2002:973:31-43).

The photo-aging mechanism will be described briefly as follows. When the skin is exposed to a large amount of UV light, a high concentration of reactive oxygen species are produced in the skin, causing the disruption of the enzymatic and non-enzymatic anti-oxidative defense systems. As a result, the content of collagen, the main protein of the skin tissue, decreases remarkably. Collagenase-1 (matrix metalloproteinase-1; MMP-1) plays an important role in decreasing the content of collagen. This enzyme is involved in the degradation of the extracellular matrix and basement membrane. According to researches, exposure to UV leads to increased MMP-1 activity in the skin, thereby markedly degrading collagen and forming wrinkles (See Sim G. S. et al., Korean J. Biotechnol. Bioeng., 2005:20(1): 40-45).

Some of active ingredients for improving wrinkles and preventing aging, which have been developed to date, have problems in that they cannot be used as cosmetic materials, are very unstable or are not easy to reach the skin. Accordingly, there exist needs for special stabilizing and delivery systems, leaving the possibility of improving skin wrinkles not visible. For these reasons, interest in skin-protecting agents containing retinoid has recently been increased. Currently, retinoid is used as a means for solving photo-aging phenomena, such as wrinkles, skin thickening, skin drooping and decrease in skin elasticity resulting from sunlight exposure. However, retinoid is such a very unstable compound, which is sensitive to UV light, moisture, heat and oxygen, that its chemical change easily occurs. In an attempt to solve this problem, studies have been primarily conducted on developing effective ingredients derived from natural resources

### SUMMARY OF THE DISCLOSURE

The inventors of the present invention have researched on natural substances which are capable of inhibiting the reduction of collagen in the skin. As a result, they have accomplished the present invention by finding out that a flavone-based compound and a Kaempferia parviflora extract comprising the same are effective in inhibiting collagen degradation, enhancing collagen synthesis and improving skin moisture.

Accordingly, one object of the present invention is to provide a cosmetic composition for wrinkle improvement, anti-aging and skin elasticity enhancement comprising one or more flavone-based compound represented by the following Chemical Formulas 1 to 3 or its salt thereof as an active ingredient:

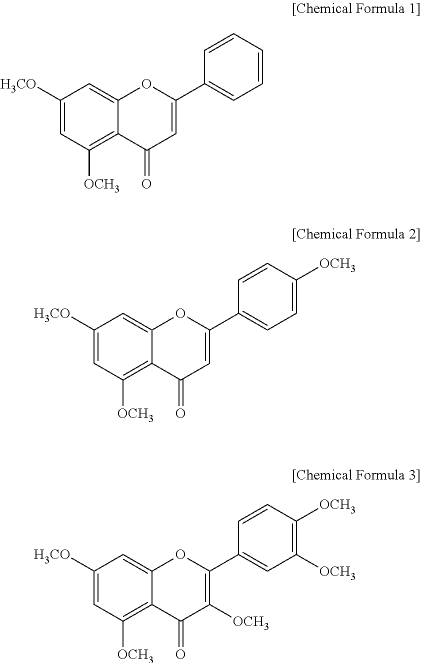 [SMILES: COc1cc(OC)c2c(=O)cc(-c3ccccc3)oc2c1 ; COc1ccc(-c2cc(=O)c3c(OC)cc(OC)cc3o2)cc1 ; COc1cc(OC)c2c(=O)c(OC)c(-c3ccc(OC)c(OC)c3)oc2c1]

Another object of the present invention is to provide a food composition for wrinkle improvement, anti-aging and skin elasticity enhancement comprising one or more flavone-based compound represented by the above-described Chemical Formulas 1 to 3 or its salt thereof as an active ingredient.

A further object of the present invention is to provide a pharmaceutical composition for wrinkle improvement, anti-aging and skin elasticity enhancement comprising one or more flavone-based compound represented by the above-described Chemical Formulas 1 to 3 or its pharmaceutically acceptable salt thereof as an active ingredient.

Another object of the present invention is to provide a cosmetic composition for skin moisturization comprising one or more flavone-based compound represented by the above-described Chemical Formulas 1 to 3 or its salt thereof as an active ingredient.

A further object of the present invention is to provide a food composition for skin moisturization comprising one or more flavone-based compound represented by the above-described Chemical Formulas 1 to 3 or its salt thereof as an active ingredient.

Another further object of the present invention is to provide a cosmetic composition for wrinkle improvement, anti-aging and skin elasticity enhancement comprising a Kaempferia parviflora extract as an active ingredient.

Another further object of the present invention is to provide a food composition for wrinkle improvement, anti-aging and skin elasticity enhancement comprising a Kaempferia parviflora extract as an active ingredient.

Another further object of the present invention is to provide a pharmaceutical composition for wrinkle improvement, anti-aging and skin elasticity enhancement comprising a Kaempferia parviflora extract as an active ingredient.

Still another object of the present invention is to provide a cosmetic composition for skin moisturization comprising a Kaempferia parviflora extract as an active ingredient.

Still another further object of the present invention is to provide a food composition for skin moisturization comprising a Kaempferia parviflora extract as an active ingredient.

Another further object of the present invention is to provide a use of one or more flavone-based compound represented by the above-described Chemical Formulas 1 to 3 or its pharmaceutically acceptable salt thereof for preparing an agent for wrinkle improvement, anti-aging and skin elasticity enhancement.

Yet another further object of the present invention is to provide a method for wrinkle improvement, anti-aging and skin elasticity enhancement comprising administering or applying to a subject in need thereof an effective amount of one or more flavone-based compound represented by the Chemical Formulas 1 to 3 or its pharmaceutically acceptable salt thereof.

Another object of the present invention is to provide a use of one or more flavone-based compound represented by the above-described Chemical Formulas 1 to 3 or its pharmaceutically acceptable salt thereof for preparing an agent for skin moisturization.

Another further object of the present invention is to provide a method for skin moisturization comprising administering or applying to a subject in need thereof an effective amount of one or more flavone-based compound represented by the Chemical Formulas 1 to 3 or its pharmaceutically acceptable salt thereof.

Another further object of the present invention is to provide a use of a Kaempferia parviflora extract for preparing an agent for wrinkle improvement, anti-aging and skin elasticity enhancement.

Still another object of the present invention is to provide a method for wrinkle improvement, anti-aging and skin elasticity enhancement comprising administering or applying to a subject in need thereof an effective amount of a Kaempferia parviflora extract.

Still another object of the present invention is to provide a use of a Kaempferia parviflora extract for preparing an agent for skin moisturization.

Yet another further object of the present invention is to provide a method for skin moisturization comprising administering or applying to a subject in need thereof an effective amount of a Kaempferia parviflora extract.

### BRIEF DESCRIPTION OF DRAWINGS

FIG. 1 illustrates a process of isolating active substances having the effect of wrinkle improvement from Kaempferia parviflora.

FIG. 2 illustrates a ¹H-NMR spectrum of 5,7-dimethoxyflavone.

FIG. 3 illustrates a ¹³C-NMR spectrum of 5,7-dimethoxyflavone.

FIG. 4 illustrates an EI/MS spectrum of 5,7-dimethoxyflavone.

FIG. 5 illustrates a ¹H-NMR spectrum of 5,7,4′-trimethoxyflavone.

FIG. 6 illustrates a ¹³C-NMR spectrum of 5,7,4′-trimethoxyflavone.

FIG. 7 illustrates an EI/MS spectrum of 5,7,4′-trimethoxyflavone.

FIG. 8 illustrates a ¹H-NMR spectrum of 3,5,7,3′,4′-pentamethoxyflavone.

FIG. 9 illustrates a ¹³C-NMR spectrum of 3,5,7,3′,4′-pentamethoxyflavone.

FIG. 10 illustrates an EI/MS spectrum of 3,5,7,3′,4′-pentamethoxyflavone.

FIG. 11 shows the effect of 5,7-dimethoxyflavone, 5,7,4′-trimethoxyflavone, 3,5,7,3′,4′-pentamethoxyflavone or the ethanol extract of Kaempferia parviflora on the inhibition of Collagenase-1 (MMP-1) activity induced by UV light.

FIG. 12 shows the skin replicas of hairless mice upon applying 5,7-dimethoxyflavone, 5,7,4′-trimethoxyflavone, 3,5,7,3′,4′-pentamethoxyflavone or the ethanol extract of Kaempferia parviflora onto the skin of the hairless mice.

FIGS. 13A-13D show the results of Rt, Rm, Rz and Ra measurement upon applying 5,7-dimethoxyflavone, 5,7,4′-trimethoxyflavone, 3,5,7,3′,4′-pentamethoxyflavone or the ethanol extract of Kaempferia parviflora onto the skin of the hairless mice. The measurement unit is μm. Rt is the distance between the highest and lowest portions on the skin surface; Rm is the maximum Rt value among Rt values of five (5) measurement areas; Rz is the mean Rt value of Rt values of five (5) measurement areas; Ra is the arithmetic mean value of surface roughness.

FIG. 14 shows the skin replicas of hairless mice upon orally administering 5,7-dimethoxyflavone, 5,7,4′-trimethoxyflavone, 3,5,7,3′,4′-pentamethoxyflavone or the ethanol extract of Kaempferia parviflora.

FIGS. 15A-15D show the results of Rt, Rm, Rz and Ra measurement upon orally administering 5,7-dimethoxyflavone, 5,7,4′-trimethoxyflavone, 3,5,7,3′,4′-pentamethoxyflavone or the ethanol extract of Kaempferia parviflora. The measurement unit is μm. Rt is the distance between the highest and lowest portions on the skin surface; Rm is the maximum Rt value among Rt values of five (5) measurement areas; Rz is the mean Rt value of Rt values of five (5) measurement areas; Ra is the arithmetic mean value of surface roughness.

### DETAILED DESCRIPTION OF THE DISCLOSURE

In order to accomplish the above described object, the present invention provides a cosmetic composition for wrinkle improvement, anti-aging and skin elasticity enhancement comprising one or more flavone-based compound represented by the following Chemical Formulas 1 to 3 or its salt thereof as an active ingredient:

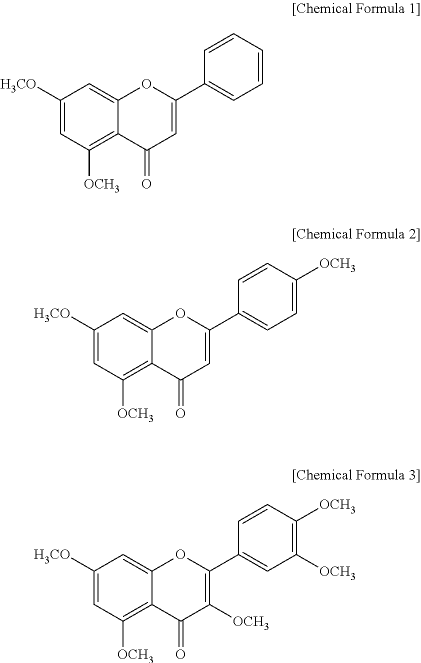 [SMILES: COc1cc(OC)c2c(=O)cc(-c3ccccc3)oc2c1 ; COc1ccc(-c2cc(=O)c3c(OC)cc(OC)cc3o2)cc1 ; COc1cc(OC)c2c(=O)c(OC)c(-c3ccc(OC)c(OC)c3)oc2c1]

In order to achieve the above described another object, the present invention provides a food composition for wrinkle improvement, anti-aging and skin elasticity enhancement comprising one or more flavone-based compound represented by the above-described Chemical Formulas 1 to 3 or its salt thereof as an active ingredient.

In order to fulfill the above described another object, the present invention provides a pharmaceutical composition for wrinkle improvement, anti-aging and skin elasticity enhancement comprising one or more flavone-based compound represented by the above-described Chemical Formulas 1 to 3 or its pharmaceutically acceptable salt thereof as an active ingredient.

In order to accomplish the above described further object, the present invention provides a cosmetic composition for skin moisturization comprising one or more flavone-based compound represented by the above-described Chemical Formulas 1 to 3 or its salt thereof as an active ingredient.

In order to achieve the above described yet further another object, the present invention provides a food composition for skin moisturization comprising one or more flavone-based compound represented by the above-described Chemical Formulas 1 to 3 or its salt thereof as an active ingredient.

In order to fulfill the above described another object, the present invention provides a cosmetic composition for wrinkle improvement, anti-aging and skin elasticity enhancement comprising a Kaempferia parviflora extract as an active ingredient.

In order to accomplish the above described yet another object, the present invention provides a food composition for wrinkle improvement, anti-aging and skin elasticity enhancement comprising a Kaempferia parviflora extract as an active ingredient.

In order to achieve the above described another further object, the present invention provides a pharmaceutical composition for wrinkle improvement, anti-aging and skin elasticity enhancement comprising a Kaempferia parviflora extract as an active ingredient.

In order to fulfill the above described still another object, the present invention provides a cosmetic composition for skin moisturization comprising a Kaempferia parviflora extract as an active ingredient.

In order to accomplish the above described another further object, the present invention provides a food composition for skin moisturization comprising a Kaempferia parviflora extract as an active ingredient.

In order to achieve the above described yet another object, the present invention provides a use of one or more flavone-based compound represented by the above-described Chemical Formulas 1 to 3 or its pharmaceutically acceptable salt thereof for preparing an agent for wrinkle improvement, anti-aging and skin elasticity enhancement.

In order to fulfill the above described another further object, the present invention provides a method for wrinkle improvement, anti-aging and skin elasticity enhancement comprising administering or applying to a subject in need thereof an effective amount of one or more flavone-based compound represented by the Chemical Formulas 1 to 3 or its pharmaceutically acceptable salt thereof.

In order to accomplish the above described another object, the present invention provides a use of one or more flavone-based compound represented by the above-described Chemical Formulas 1 to 3 or its pharmaceutically acceptable salt thereof for preparing an agent for skin moisturization.

In order to achieve the above described yet another object, the present invention provides a method for skin moisturization comprising administering or applying to a subject in need thereof an effective amount of one or more flavone-based compound represented by the Chemical Formulas 1 to 3 or its pharmaceutically acceptable salt thereof.

In order to fulfill the above described still another object, the present invention provides a use of a Kaempferia parviflora extract for preparing an agent for wrinkle improvement, anti-aging and skin elasticity enhancement.

In order to accomplish the above described another object, the present invention provides a method for wrinkle improvement, anti-aging and skin elasticity enhancement comprising administering or applying to a subject in need thereof an effective amount of a Kaempferia parviflora extract.

In order to achieve the above described another further object, the present invention provides a use of a Kaempferia parviflora extract for preparing an agent for skin moisturization.

In order to fulfill the above described yet another further object, the present invention provides a method for skin moisturization comprising administering or applying to a subject in need thereof an effective amount of a Kaempferia parviflora extract.

Hereinafter, the present invention will be described in detail.

The composition of the present invention, which can be used for wrinkle improvement, anti-aging, skin elasticity enhancement or skin moisturization, comprises one or more flavone-based compound represented by the following Chemical Formulas 1 to 3 or its salt thereof or a Kaempferia parviflora extract as an active ingredient.

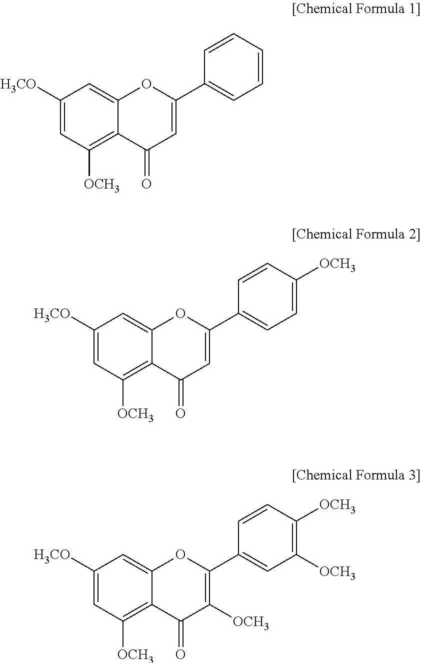 [SMILES: COc1cc(OC)c2c(=O)cc(-c3ccccc3)oc2c1 ; COc1ccc(-c2cc(=O)c3c(OC)cc(OC)cc3o2)cc1 ; COc1cc(OC)c2c(=O)c(OC)c(-c3ccc(OC)c(OC)c3)oc2c1]

The compound of Chemical Formula 1 is 5,7-dimethoxyflavone, the compound of Chemical Formula 2 is 5,7,4′-trimethoxyflavone, and the compound of Chemical Formula 3 is 3,5,7,3′,4′-pentamethoxyflavone. All of the above compounds are flavone-based compounds.

In the composition of the present invention, 5,7-dimethoxyflavone, 5,7,4′-trimethoxyflavone and 3,5,7,3′,4′-pentamethoxyflavone may be prepared according to chemical synthesis methods or extracted and subsequently isolated from Kaempferia parviflora or other plants. In the composition of the present invention, a Kaempferia parviflora extract refers to an extract obtained from Kaempferia parviflora.

Kaempferia parviflora, also known as Black ginger, is a plant of the Zingiberaceae family. The Kaempferia parviflora extract of the present invention contains flavone-based compounds, especially a large amount of 5,7-dimethoxyflavone, 5,7,4′-trimethoxyflavone and 3,5,7,3′,4′-pentamethoxyflavone (Hereinafter, 5,7-dimethoxyflavone, 5,7,4′-trimethoxyflavone and 3,5,7,3′,4′-pentamethoxyflavone as represented by Chemical Formulas 1-3 will be generally described as “flavone-based compounds of the present invention”).

The Kaempferia parviflora extract of the present invention can be obtained according to known extraction methods for natural substances, preferably by using at least one solvent which may be selected from the group consisting of water, C₁-C₆ organic solvent, and subcritical or supercritical fluid. The C₁-C₆ organic solvent may be selected from the group consisting of C₁-C₆ alcohol, acetone, ether, benzene, chloroform, ethyl acetate, methylene chloride, hexane, cyclohexane, and petroleum ether.

Preferably, the flavone-based compounds or the Kaempferia parviflora extract of the present invention may be prepared by extracting and purifying the dried rhizome of Kaempferia parviflora with solvents suitable for food processing such as water, ethanol, and subcritical or supercritical carbon dioxide. Alternatively, the flavone-based compounds or the Kaempferia parviflora extract of the present invention may be preferably prepared by separating and purifying oils obtained by direct compression of the Kaempferia parviflora plant.

More preferably, the Kaempferia parviflora extract of the present invention may be the ethanol extract of Kaempferia parviflora, while the flavone-based compounds of the present invention may be isolated from Kaempferia parviflora by using ethanol as a solvent.

Preferably, the flavone-based compounds of the present invention may be extracted from Kaempferia parviflora. For the purpose of separation and purification of the flavone-based compounds of the present invention from Kaempferia parviflora, column chromatography and/or high-performance liquid chromatography (HPLC) using silica gel, activated alumina or other various synthetic resins may be used alone or in combination, although not limited thereto.

For the isolation of 5,7-dimethoxyflavone among the flavone-based compounds according to the present invention, the ethanol extract of Kaempferia parviflora may be placed in a column in which silica gel is packed, and subsequently separated by using a solvent system containing a mixture of ethyl acetate and methanol (10:0.5, v/v), leading to a total of five (5) fractions. Out of the five fractions, the 3rd fraction may be subjected to further separation through Rp-18 Column Chromatography using 70% methanol, leading to a total of two (2) fractions. Out of the two fractions, the 2^{nd} fraction may be again placed in a column in which silica gel is packed, and subsequently separated by using a solvent system containing a mixture of ethyl acetate and methanol (10:0.4, v/v), leading to a total of two (2) fractions. Out of the two fractions, the 1^{st} fraction may be concentrated and dried to produce 5,7-dimethoxyflavone of the present invention.

For the isolation of 5,7,4′-trimethoxyflavone among the flavone-based compounds according to the present invention, the ethanol extract of Kaempferia parviflora may be placed in a column in which silica gel is packed, and subsequently separated by using a solvent system containing a mixture of ethyl acetate and methanol (10:0.5, v/v), leading to a total of five (5) fractions. Out of the five fractions, the 4^{th} fraction may be subjected to further separation through Rp-18 Column Chromatography using 70% methanol, leading to a total of two (2) fractions. Out of the two fractions, the 2^{nd} fraction may be concentrated and dried to produce 5,7,4′-trimethoxyflavone of the present invention.

For the isolation of 3,5,7,3′,4′-pentamethoxyflavone among the flavone-based compounds according to the present invention, the ethanol extract of Kaempferia parviflora may be placed in a column in which silica gel is packed, and subsequently separated by using a solvent system containing a mixture of ethyl acetate and methanol (10:0.5, v/v), leading to a total of five (5) fractions. Out of the five fractions, the 3^{rd} fraction may be subjected to further separation through Rp-18 Column Chromatography using 70% methanol, leading to a total of two (2) fractions. Out of the two fractions, the 1^{st} fraction may be concentrated and dried to produce 3,5,7,3′,4′-pentamethoxyflavone of the present invention.

The flavone-based compounds or the Kaempferia parviflora extract of the present invention are excellent in inhibiting collagen degradation and promoting collagen synthesis.

In one embodiment of the present invention, the flavone-based compounds or the Kaempferia parviflora extract according to the present invention were tested to see whether or not they may possess inhibitory activity of suppressing the synthesis of Collagenase-1 (MMP-1) after exposure to UV light and subsequent activation of Collagenase-1 production in human fibroblast cells. The test results showed that the flavone-based compounds or the Kaempferia parviflora extract according to the present invention effectively suppressed the synthesis of Collagenase-1 (MMP-1).

In another embodiment of the present invention, the degree of collagen synthesis was measured after the flavone-based compounds or the Kaempferia parviflora extract according to the present invention were applied to human fibroblast cells. The test results showed that the flavone-based compounds or the Kaempferia parviflora extract according to the present invention were excellent in promoting collagen synthesis which was suppressed by the exposure to UV light.

Increased activation of MMP-1 and subsequent degradation of collagen, which is the main component of the skin tissue, may induce wrinkle formation, accelerate aging and decrease skin elasticity.

The composition of the present invention may suppress wrinkle formation, inhibit aging and improve skin elasticity by suppressing the activity of MMP-1 and enhancing the synthesis of collagen.

Further, the composition of the present invention is effective in skin moisturization by inhibiting the moisture loss in the skin.

In one embodiment of the present invention, the composition of the present invention was applied onto the skin or administered orally to UV photo-aging induced mice, followed by observing the change in their skin condition. The results showed the improvement of wrinkles and the enhancement of skin elasticity in a group of mice to which the composition of the present invention was applied onto the skin or administered orally. It was also observed that the amount of moisture loss in the skin was significantly reduced.

As described above, the flavone-based compounds or the Kaempferia parviflora extract according to the present invention may be effectively used in cosmetic compositions, foods, dietary supplements, pharmaceutical products and the like for wrinkle improvement, anti-aging, skin elasticity enhancement and skin moisturization.

Hence, the present invention provides a cosmetic composition, a food composition and a pharmaceutical composition for wrinkle improvement, anti-aging and skin elasticity enhancement comprising one or more flavone-based compound according to the present invention as an active ingredient.

Further, the present invention provides a cosmetic composition and a food composition for skin moisturization comprising one or more flavone-based compound according to the present invention as an active ingredient.

Furthermore, the present invention provides a cosmetic composition, a food composition and a pharmaceutical composition for wrinkle improvement, anti-aging and skin elasticity enhancement comprising a Kaempferia parviflora extract according to the present invention as an active ingredient.

Still furthermore, the present invention provides a cosmetic composition and a food composition for skin moisturization comprising a Kaempferia parviflora extract according to the present invention as an active ingredient.

The cosmetic composition of the present invention may be prepared in any type of formulation commonly used in the cosmetic industry. It may be prepared in the form of additives for local or systemic application as commonly used in the dermatology, while further comprising dermatologically acceptable medium or base in addition to a Kaempferia parviflora extract or flavone-based compounds according to the present invention.

Further, while the cosmetic composition of the present invention comprises a Kaempferia parviflora extract or flavone-based compounds according to the present invention, it may further comprises, but not limited thereto, fats, organic solvents, solubilizers, thickeners, gelants, emollients, anti-oxidants, suspending agents, stabilizers, foaming agents, flavoring agents, surfactants, water, ionic or non-ionic emulsifiers, filing agents, sequestrants, chelating agents, preservatives, vitamins, blockers, humectants, essential oils, pigments, coloring agents, hydrophilic or lipophilic activators, lipid vesicles or other commonly used excipients in cosmetics or dermatology industries. The cosmetic composition according to the present invention may be suitably formulated in the form of, for example, solution, gel, solid, anhydrous paste, oil-in-water emulsion, suspension, micro-emulsion, micro-capsule, micro-granule, ionic (liposome) or non-ionic vesicular dispersion agent, cream, skin lotion, lotion, powder, ointment, spray or conceal stick. Further, it may be prepared in the form of foam or aerosol further comprising condensed propellant.

The cosmetic composition according to the present invention may be contained in cosmetic products including, but not limited thereto, skin lotion, skin softener, skin toner, astringent lotion, emollient lotion, nutritional lotion, astringent, lotion, milk lotion, moisture lotion, nutritional lotion, body cream, massage cream, nutritional cream, moisture cream, hand cream, essence, nutritional essence, pack, soap, shampoo, cleansing foam, cleansing lotion, cleansing cream, body lotion, body cleanser, treatment, cosmetic liquid, emulsion, pressed powder, loose powder and eye shadow.

The Kaempferia parviflora extract or flavone-based compounds according to the present invention as contained in the cosmetic composition of the present invention may be included in the range of 0.0001-50 wt %, and preferably 0.01-10 wt %, based on the total weight of the cosmetic composition, while carriers or additives may account for the rest of the total weight.

The food composition of the present invention may be provided in all types of forms including functional food, nutritional supplement, health food, and food additives. The said food composition may be prepared into various kinds of forms by the usual methods known in the art.

For example, as a health food, the Kaempferia parviflora extract or flavone-based compounds according to the present invention may be prepared into tea, juice or drink for drinking, or into granules, capsules or powders for ingestion. In addition, the food composition of the present invention may be prepared by mixing the Kaempferia parviflora extract or flavone-based compounds according to the present invention along with conventional active ingredients which are well known as being effective in wrinkle improvement, anti-aging, skin elasticity enhancement and skin moisturization.

Further, for preparing a functional food, the Kaempferia parviflora extract or flavone-based compounds according to the present invention may be added to, but not limited thereto, beverages (including alcoholic beverages), fruits and their processed foods (e.g. canned fruit, bottled fruit, jam, marmalade etc.), fishes, meats and their processed foods (e.g. ham, sausage, corn beef etc.), breads and noodles (e.g. Japanese noodle, buckwheat noodle, ramen, spaghetti, macaroni etc.), fruit juice, various drinks, cookies, taffy, dairy products (e.g. butter, cheese etc.), vegetable oil, margarine, vegetable protein, retort food, frozen food or various seasonings (e.g. soybean paste, soy sauce, sauce etc.).

Furthermore, the Kaempferia parviflora extract or flavone-based compounds according to the present invention may be prepared in the form of powder or concentrate for their use as a food additive.

The Kaempferia parviflora extract or flavone-based compounds according to the present invention as contained in the food composition of the present invention may be included in the range of 0.0001-50 wt %, and preferably 0.01-10 wt %, based on the total weight of the food composition, while carriers or additives may account for the rest of the total weight.

The Kaempferia parviflora extract or flavone-based compounds according to the present invention may be used in their natural form or in the form of salts or pharmaceutically acceptable salts thereof. The term “pharmaceutically acceptable” means being physiologically acceptable without causing allergic or similar reactions upon administering to human. The pharmaceutically acceptable salts are preferably acid addition salts derived from pharmaceutically acceptable free acids. The free acids may be inorganic or organic acids. The organic acids include, but not limited thereto, citric acid, acetic acid, lactic acid, tartaric acid, maleic acid, fumaric acid, formic acid, propionic acid, oxalic acid, trifluoroacetic acid, benzoic acid, gluconic acid, meta sulfonic acid, glycolic acid, succinic acid, 4-toluenesulfonic acid, glutamic acid and aspartic acid. The inorganic acids include, but not limited thereto, hydrochloric acid, bromic acid, sulfuric acid and phosphoric acid.

The pharmaceutical composition of the present invention may comprise a pharmaceutically effective amount of the Kaempferia parviflora extract, flavone-based compounds according to the present invention or salts thereof alone or in combination with one or more pharmaceutically acceptable carrier, excipient or diluent. The term “pharmaceutically acceptable” means being physiologically acceptable and non-toxic without causing allergic or similar reactions (such as gastroenteric trouble and dizziness) upon administering to human, while not adversely affecting the effect of the active ingredients in the composition.

The pharmaceutically acceptable carrier, excipient and diluent may include, for example, lactose, dextrose, sucrose, sorbitol, mannitol, xylitol, erythritol, maltitol, starch, acacia rubber, alginate, gelatin, calcium phosphate, calcium silicate, cellulose, methyl cellulose, polyvinylpyrrolidone, water, methylhydroxybenzoate, propylhydroxybenzoate, talc, magnesium stearate and mineral oil. Further, the pharmaceutical composition of the present invention may additionally comprises, but not limited thereto, fillers, anti-agglomerating agents, lubricants, humectants, flavoring agents, emulsifiers and preservatives.

The term “pharmaceutically effective amount” means an amount which produces the superior effect to that of negative control groups, preferably an amount which is sufficient to produce wrinkle improvement, anti-aging and skin elasticity enhancement. The pharmaceutically effective amount of the Kaempferia parviflora extract or flavone-based compounds according to the present invention is 0.01 to 200 mg/day/kg body weight. However, the pharmaceutically effective amount may be suitably determined by considering various factors, such as diseases, the severity of diseases, age, body weight, health condition, gender, administration route and treatment period.

Furthermore, the pharmaceutical composition of the present invention may be formulated by using known methods in the art into a form for immediate-, sustained- or delayed release of the active ingredients in the composition upon administering to mammals. It may be formulated into powders, granules, tablets, emulsions, syrups, aerosols, soft or hard gelatin capsules, sterilized injection solution or sterilized powders.

The pharmaceutical composition of the present invention may be administered by various routes including, but not limited thereto, oral or parenteral routes. The parenteral routes include, but not limited thereto, percutaneous, intranasal, intraperitoneal, intramuscular, subcutaneous or intravenous routes.

The pharmaceutical composition of the present invention may be administered in combination with known compounds effective in wrinkle improvement, anti-aging and skin elasticity enhancement.

As described above, the present invention provides a cosmetic composition, a food composition and a pharmaceutical composition for wrinkle improvement, anti-aging and skin elasticity enhancement or skin moisturization comprising the Kaempferia parviflora extract or flavone-based compounds according to the present invention as an active ingredient. The composition of the present invention is effective in wrinkle improvement, anti-aging and skin elasticity enhancement by inhibiting the activity of MMP-1 and promoting the synthesis of collagen, while being further effective in skin moisturization by decreasing moisture loss from the skin.

Hereinafter, the present invention will be described in detail through the following examples. However, the following examples are given only for the purpose of illustrating the present invention, and the scope of the present invention is not limited by them in any way.

### Example 1

### Preparation of the Ethanol Extract of Kaempferia parviflora

Kaempferia parviflora rhizome was ground using a mixer. 100 g of the ground Kaempferia parviflora sample was then added to 1 L of ethanol and extracted after macerating at room temperature for 48 hours. The extracted sample was filtered through Whatman No. 2 filter paper. The solvent component was then removed from the filtered extract solution by concentrating using a rotary vacuum evaporator, resulting in obtaining the ethanol extract of Kaempferia parviflora.

### Example 2

### Isolation and Identification of 5,7-dimethoxyflavone

<2-1> Isolation of 5,7-Dimethoxyflavone

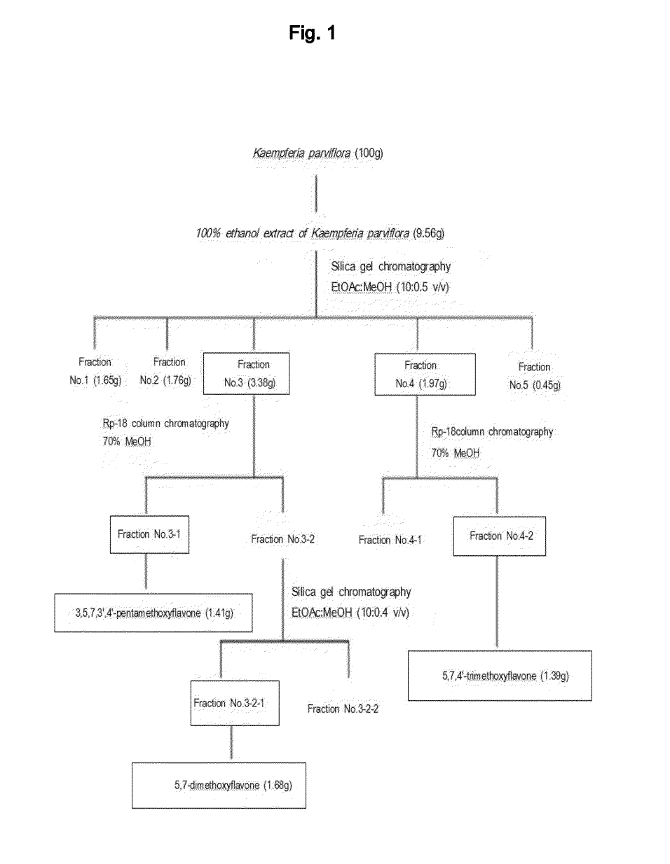

The concentrated ethanol extract of Kaempferia parviflora obtained in Example 1 was placed in a column (6×15 cm) packed with silica gel, and subsequently separated by using a solvent system containing a mixture of ethyl acetate and methanol (10:0.5, v/v). A total of five (5) fractions were obtained in the order of separation, followed by concentration drying. Out of the five fractions, the 3rd fraction (Fraction No. 3) was subjected to further separation through Rp-18 Column Chromatography (Lichroprep® RP-18 25˜40 μm, Merck & Co., USA) using 70% methanol as a developing solvent. A total of two (2) fractions were obtained in the order of separation, followed by concentration drying. Out of the two fractions, the 2^{nd} fraction (Fraction No. 3-2) was placed in a column (6×15 cm) packed with silica gel, and subsequently separated by using a solvent system containing a mixture of ethyl acetate and methanol (10:0.4, v/v), leading to a total of two (2) fractions. Out of the two fractions, the 1^{st} fraction (Fraction No. 3-2-1) was concentrated and dried to isolate a pure active substance effective for wrinkle improvement. Procedures of the above described isolation process are illustrated in FIG. 1.

<2-2> Identification of the Chemical Structure of 5,7-Dimethoxyflavone

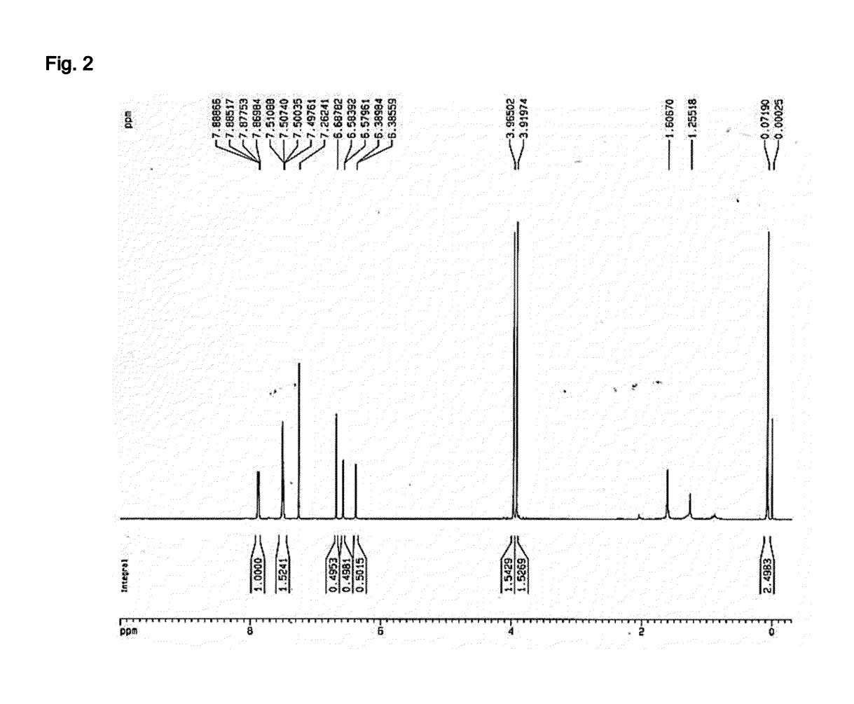

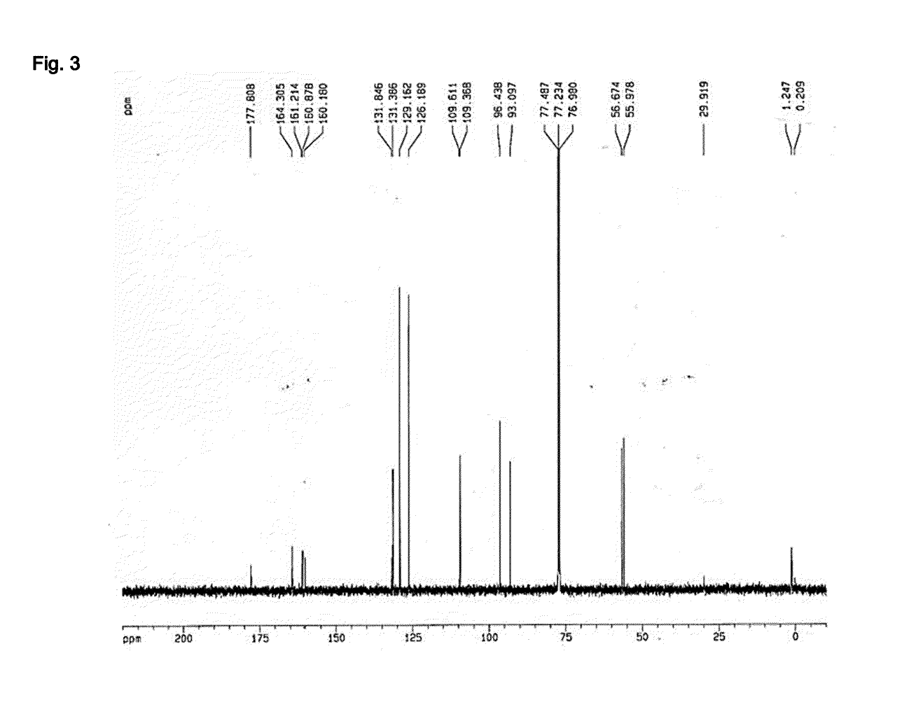

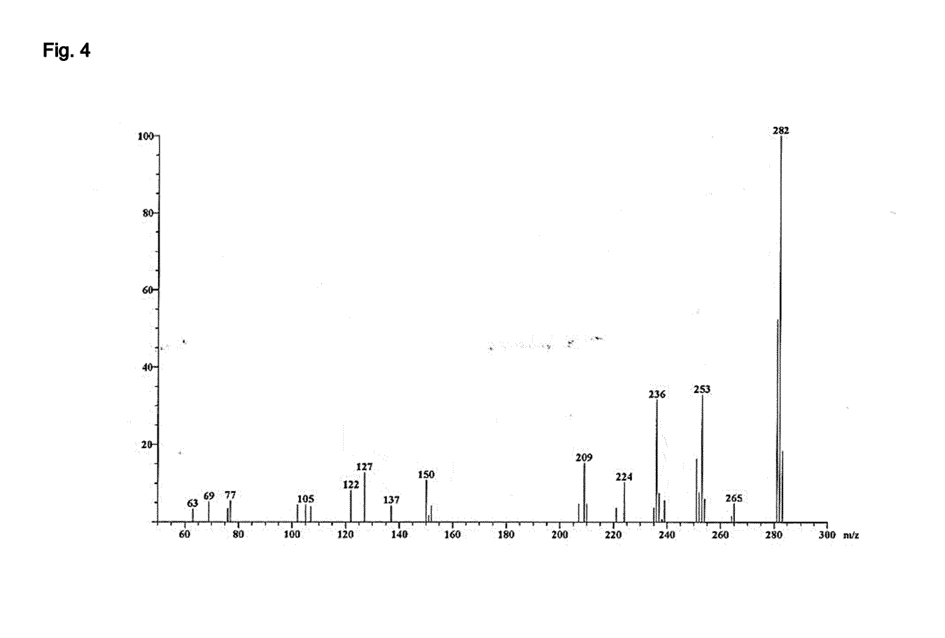

In order to determine the chemical structure of the pure active substance as isolated in Example <2-1>, ¹H-NMR spectrum and ¹³C-NMR spectrum were observed at 500 MHz and 125 MHz (solvent: CDCl₃), respectively. The results of ¹H-NMR spectrum and ¹³C-NMR spectrum are shown in FIGS. 2 & 3. Further, for mass analysis of the isolated pure substance, the result of EI/MS is illustrated in FIG. 4. The molecular weight of the said compound was found 282 since [M] was detected at m/z 282 in EI/MS. Upon analyzing the results of ¹H-NMR, ¹³C-NMR and EI/MS in comparison with the prior research data (Sutthanut K. et al., J. Chromatography A, 2007:1143:227-233), the above isolated pure substance was identified as 5,7-dimethoxyflavone represented by the following Chemical Formula 1.

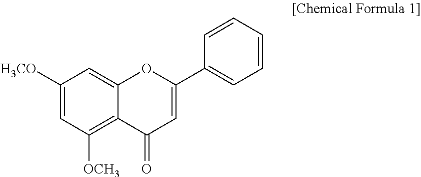 [SMILES: COc1cc(OC)c2c(=O)cc(-c3ccccc3)oc2c1]

### Example 3

### Isolation and Identification of 5,7,4′-trimethoxyflavone

<3-1> Isolation of 5,7,4′-Trimethoxyflavone

The concentrated ethanol extract of Kaempferia parviflora obtained in Example 1 was placed in a column (6×15 cm) packed with silica gel, and subsequently separated by using a solvent system containing a mixture of ethyl acetate and methanol (10:0.5, v/v). A total of five (5) fractions were obtained in the order of separation, followed by concentration drying. Out of the five fractions, the 4^{th} fraction (Fraction No. 4) was subjected to further separation through Rp-18 Column Chromatography (Lichroprep® RP-18 25˜40 μm, Merck & Co., USA) using 70% methanol as a developing solvent. A total of two (2) fractions were obtained in the order of separation, followed by concentration drying. Out of the two fractions, the 2^{nd} fraction (Fraction No. 4-2) was concentrated and dried to isolate a pure active substance effective for wrinkle improvement. Procedures of the above described isolation process are illustrated in FIG. 1.

<3-2> Identification of the Chemical Structure of 5,7,4′-Trimethoxyflavone

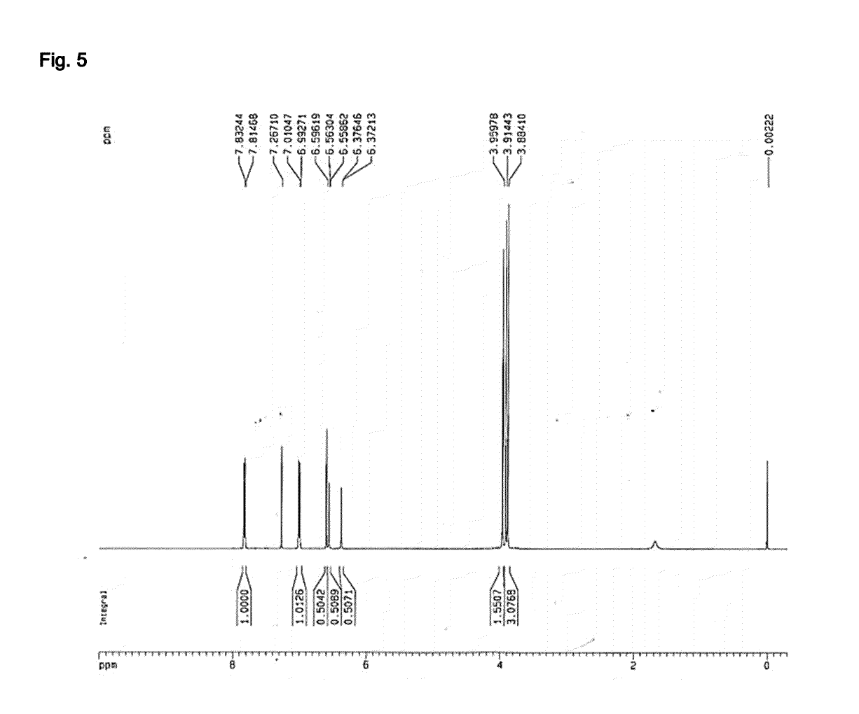

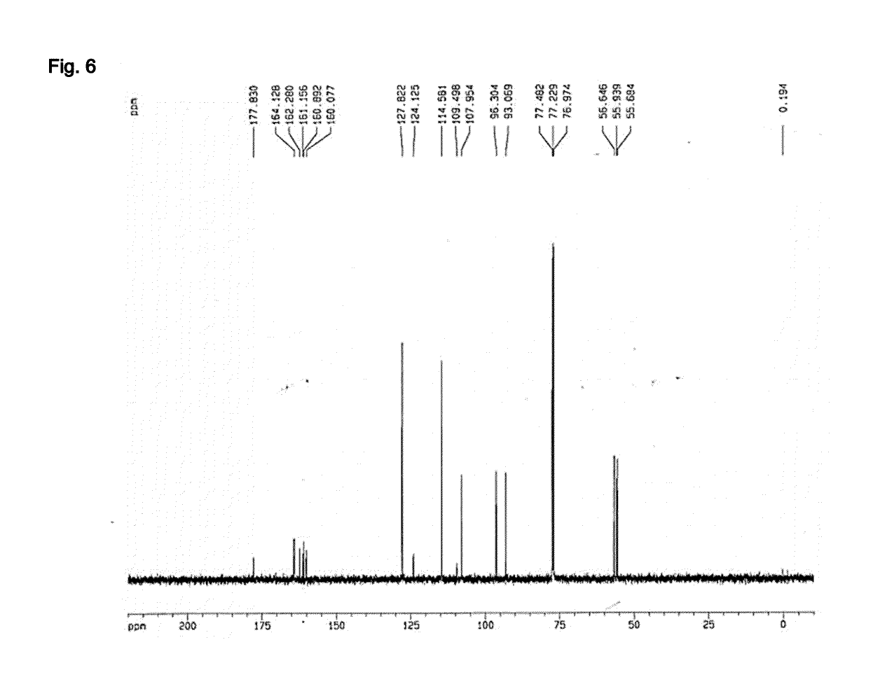

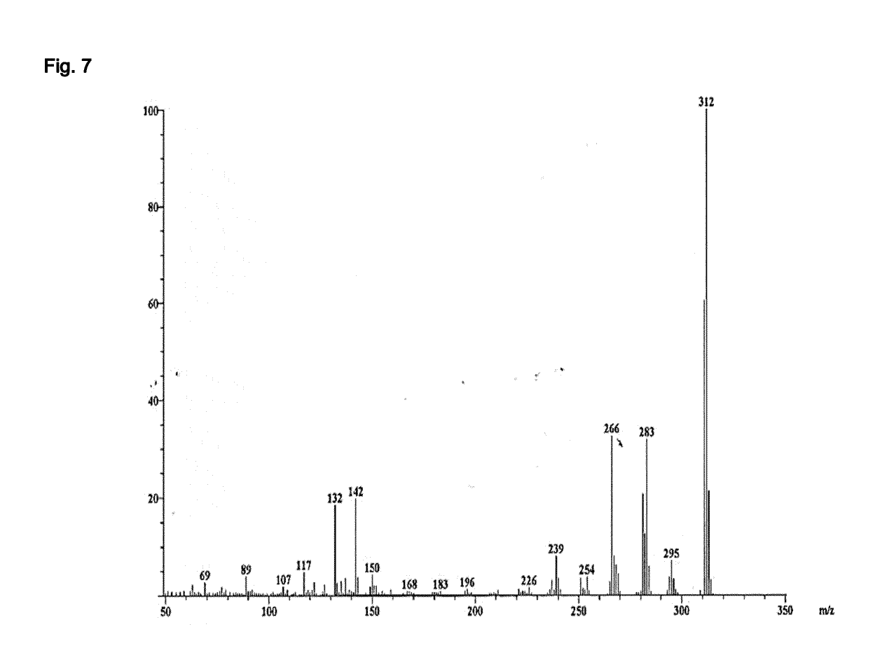

In order to determine the chemical structure of the pure active substance as isolated in Example <3-1>, ¹H-NMR spectrum and ¹³C-NMR spectrum were observed at 500 MHz and 125 MHz (solvent: CDCl₃), respectively. The results of ¹H-NMR spectrum and ¹³C-NMR spectrum are shown in FIGS. 5 & 6. Further, for mass analysis of the isolated pure substance, the result of EI/MS is illustrated in FIG. 7. The molecular weight of the said compound was 312 since [M] was detected at m/z 312 in EI/MS. Upon analyzing the results of ¹H-NMR, ¹³C-NMR and EI/MS in comparison with the prior research data (Sutthanut K. et al., J. Chromatography A, 2007:1143:227-233), the above isolated pure substance was identified as 5,7,4′-trimethoxyflavone represented by the following Chemical Formula 2.

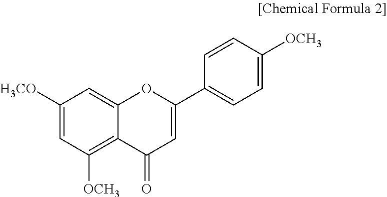 [SMILES: COc1ccc(-c2cc(=O)c3c(OC)cc(OC)cc3o2)cc1]

### Example 4

### Isolation and Identification of 3,5,7,3′,4′-pentamethoxyflavone

<4-1> Isolation of 3,5,7,3′,4′-Pentamethoxyflavone

The concentrated ethanol extract of Kaempferia parviflora obtained in Example 1 was placed in a column (6×15 cm) packed with silica gel, and subsequently separated by using a solvent system containing a mixture of ethyl acetate and methanol (10:0.5, v/v). A total of five (5) fractions were obtained in the order of separation, followed by concentration drying. Out of the five fractions, the 3^{rd} fraction (Fraction No. 3) was subjected to further separation through Rp-18 Column Chromatography (Lichroprep® RP-18 25˜40 μm, Merck & Co., USA) using 70% methanol as a developing solvent. A total of two (2) fractions were obtained in the order of separation, followed by concentration drying. Out of the two fractions, the 1^{st} fraction (Fraction No. 3-1) was concentrated and dried to isolate a pure active substance effective for wrinkle improvement. Procedures of the above described isolation process are illustrated in FIG. 1.

<4-2> Identification of the Chemical Structure of 3,5,7,3′,4′-Pentamethoxyflavone

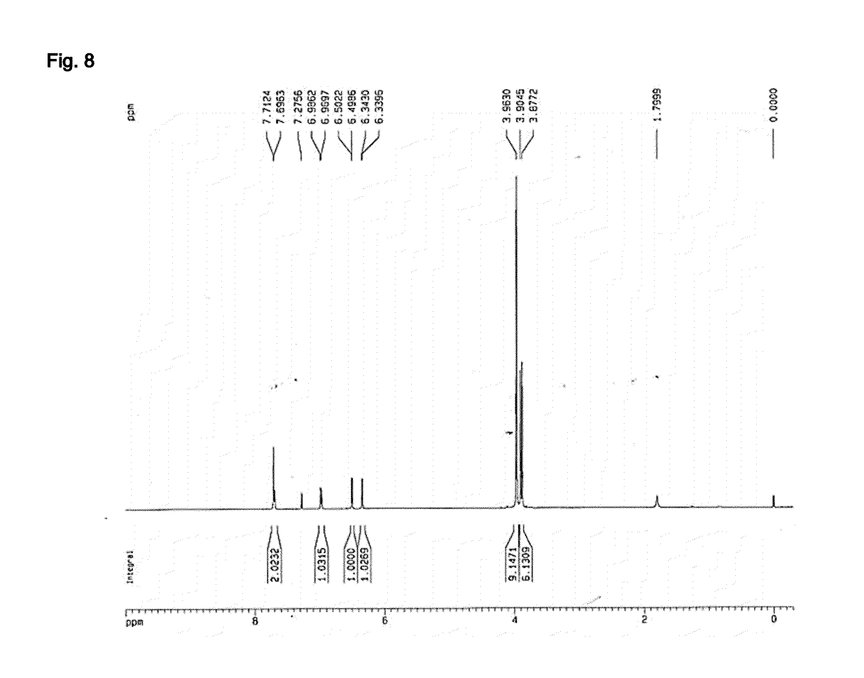

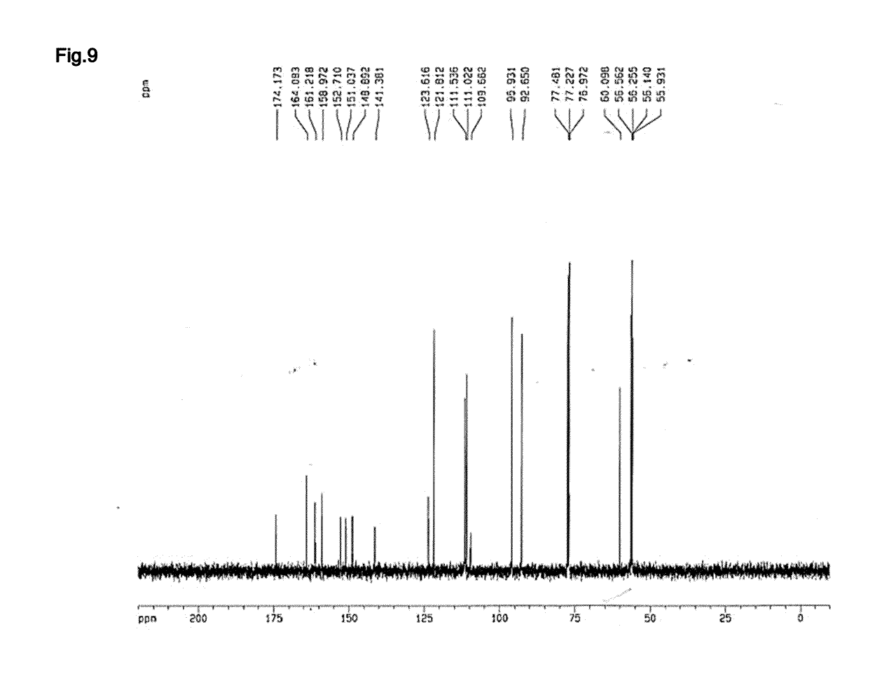

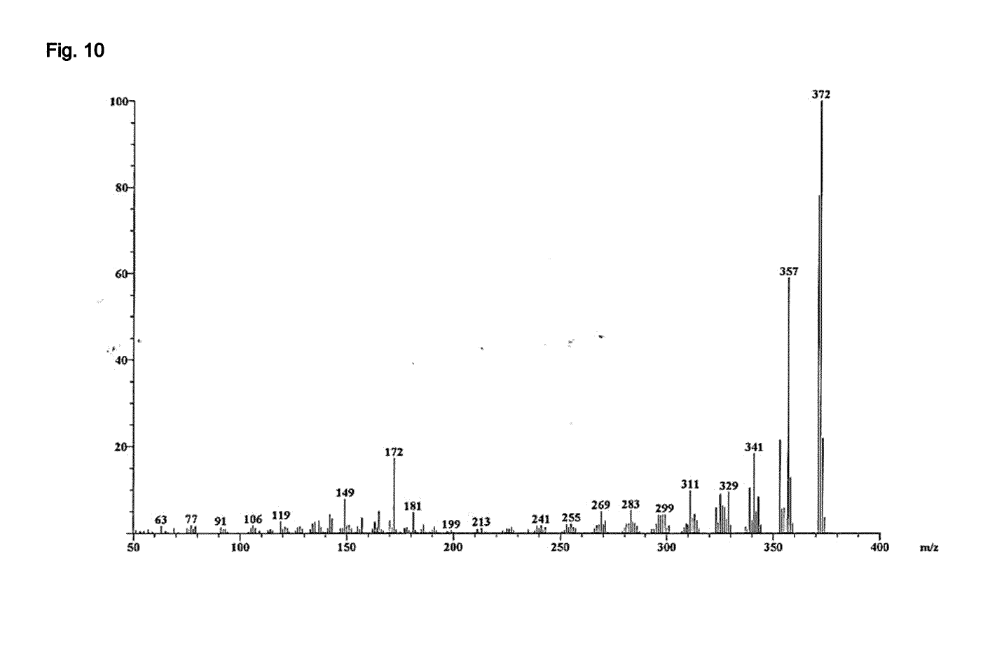

In order to determine the chemical structure of the pure active substance as isolated in Example <4-1>, ¹H-NMR spectrum and ¹³C-NMR spectrum were observed at 500 MHz and 125 MHz (solvent: CDCl₃), respectively. The results of ¹H-NMR spectrum and ¹³C-NMR spectrum are shown in FIGS. 8 & 9. Further, for mass analysis of the isolated pure substance, the result of EI/MS is illustrated in FIG. 10. The molecular weight of the said compound was found 372 since NI was detected at m/z 372 in EI/MS. Upon analyzing the results of ¹H-NMR, ¹³C-NMR and EI/MS in comparison with the prior research data (Sutthanut K. et al., J. Chromatography A, 2007:1143:227-233), the above isolated pure substance was identified as 3,5,7,3′,4′-pentamethoxyflavone represented by the following Chemical Formula 3.

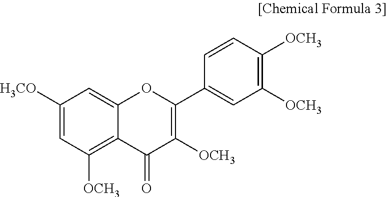 [SMILES: COc1cc(OC)c2c(=O)c(OC)c(-c3ccc(OC)c(OC)c3)oc2c1]

### Example 5

### Inhibition of UV-Induced Collagenase-1 Activity

5,7-dimethoxyflavone, 5,7,4′-trimethoxyflavone, 3,5,7,3′,4′-pentamethoxyflavone and the ethanol extract of Kaempferia parviflora as obtained in Examples 1-4 were tested to see whether they possess an inhibitory activity on Collagenase-1 (Matrix Metalloproteinase-1, MMP-1).

Firstly, human fibroblasts were placed in a 96-well microtiter plate (5,000 cells/well) containing DMEM (Dulbecco's Modified Eagle's Media) which comprises 2.5% bovine fetal serum, and cultured until they reached a growth rate of around 90%. Then, the cells were treated for 24 hours in serum-free DMEM containing 20 μM of 5,7-dimethoxyflavone, 20 μM 5,7,4′-trimethoxyflavone, 20 μM 3,5,7,3′,4′-pentamethoxyflavone and 20 μg/ml of the ethanol extract of Kaempferia parviflora of Examples 1-4, respectively, and followed by the exposure to 15 mJ of UV. Subsequently, the cells were additionally treated with serum-free DMEM containing 20 μM of 5,7-dimethoxyflavone, 20 μM 5,7,4′-trimethoxyflavone, 20 μM 3,5,7,3′,4′-pentamethoxyflavone and 20 μg/ml of the ethanol extract of Kaempferia parviflora of Examples 1-4, respectively. After 24 hours, the cell culture was collected and centrifuged to obtain the supernatant.

For the resulting supernatant, the degree of Collagenase-1 (MMP-1) production was observed by Collagenase-1 Measurement Kit (QIA55, Merch & Co., USA). Firstly, the above obtained cell culture was placed in a 96-well plate evenly coated with primary antibodies against Collagenase-1 (MMP-1), followed by antigen-antibody reaction for 2 hours at room temperature. After 2 hours, primary antibodies against collagen with fluorescence were added to the said 96-well plate for 1 hour. After 1 hour, a substance inducing color formation was added and left for 30 minutes at room temperature. Then, a termination buffer was added to discontinue the color formation, resulting in the formation of yellow color. The degree of yellow color varied depending on the degree of the said reaction process. Absorbance of the 96-well plate showing yellow color was measured with an absorptiometer at 450 nm.

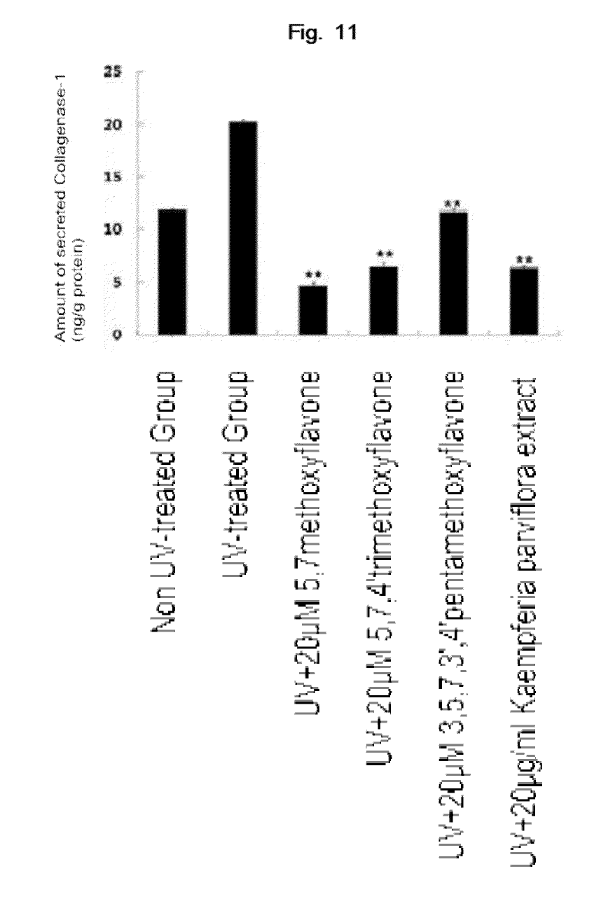

As shown in FIG. 11, 5,7-dimethoxyflavone, 5,7,4′-trimethoxyflavone, 3,5,7,3′,4′-pentamethoxyflavone or the ethanol extract of Kaempferia parviflora were excellent for inhibiting Collagenase-1 (MMP-1) activity.

### Example 6

### Enhancing Effect of Collagen Production

5,7-dimethoxyflavone, 5,7,4′-trimethoxyflavone, 3,5,7,3′,4′-pentamethoxyflavone and the ethanol extract of Kaempferia parviflora as obtained in Examples 1-4 were tested to see whether they possess an enhancing activity on collagen production.

Firstly, human fibroblasts were placed in a 96-well microtiter plate (5,000 cells/well) containing DMEM (Dulbecco's Modified Eagle's Media) which comprises 2.5% bovine fetal serum, and cultured until they reached a growth rate of around 90%. Then, the cells were treated for 24 hours in serum-free DMEM containing 20 μM of 5,7-dimethoxyflavone, 20 μM 5,7,4′-trimethoxyflavone, 20 μM 3,5,7,3′,4′-pentamethoxyflavone and 20 μg/ml of the ethanol extract of Kaempferia parviflora of Examples 1-4, respectively, and followed by the exposure to 15 mJ of UV. Subsequently, the cells were additionally treated with serum-free DMEM containing 20 μM of 5,7-dimethoxyflavone, 20 μM 5,7,4′-trimethoxyflavone, 20 μM 3,5,7,3′,4′-pentamethoxyflavone and 20 μg/ml of the ethanol extract of Kaempferia parviflora of Examples 1-4, respectively. After 24 hours, the cell culture was collected and centrifuged to obtain the supernatant. For the obtained supernatant, the production of Procollagen Type-I C Peptide (PIP), an indicator of collagen production, was quantitatively measured by ELISA kit (MK101, Takara Bio Ink., Japan) so as to observe their effect on collagen production.

Firstly, antibody-PIP conjugates tagged to peroxidase were added to a 96-well plate evenly coated with mouse monoclonal antibodies against Procollagen. Then, the above obtained cell culture was added and reacted for 3 hours in an incubator, followed by the addition of substrate solution for color formation. Upon staying for 15 minutes at room temperature, the reaction was ended after the addition of reaction termination solution. Absorbance was measured with an absorptiometer at 450 nm.

As shown in the following Table 1,5,7-dimethoxyflavone, 5,7,4′-trimethoxyflavone, 3,5,7,3′,4′-pentamethoxyflavone or the ethanol extract of Kaempferia parviflora were excellent for enhancing collagen production.

TABLE 1
Test result for the enhancing effect of collagen production

| | Content of Collagen |
|---|---|
| Test Groups | (ng/10⁵ cell) |
| Non UV-treated Group | 258.0 |
| UV-treated Group | 179.4 |
| UV+ 7-dimethoxyflavone-treated Group | 236.7 |
| UV+ 5,7,4′-trimethoxyflavone-treated Group | 230.0 |
| UV+ 3,5,7,3′,4′-pentamethoxyflavone-treated | 226.5 |
| Group | |
| UV+ the ethanol extract of Kaempferia | 224.4 |
| parviflora - treated Group | |

### Example 7

### In Vivo Improvement on UV-Induced Wrinkles

### Example 7-1

### Application onto the Skin

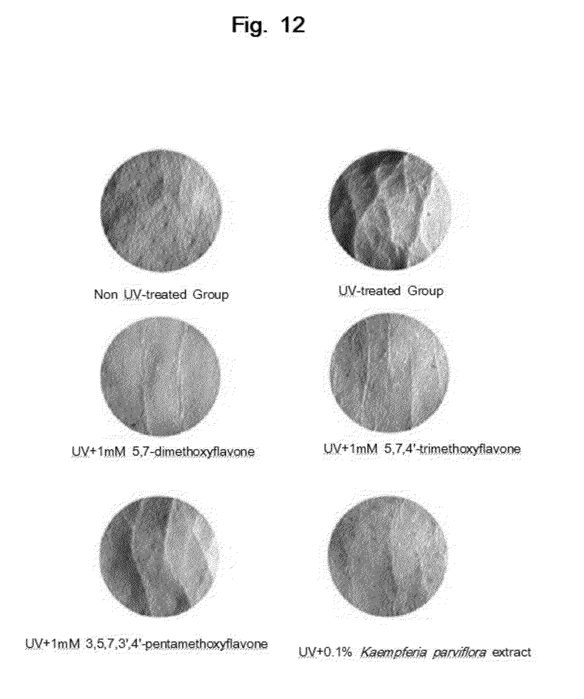

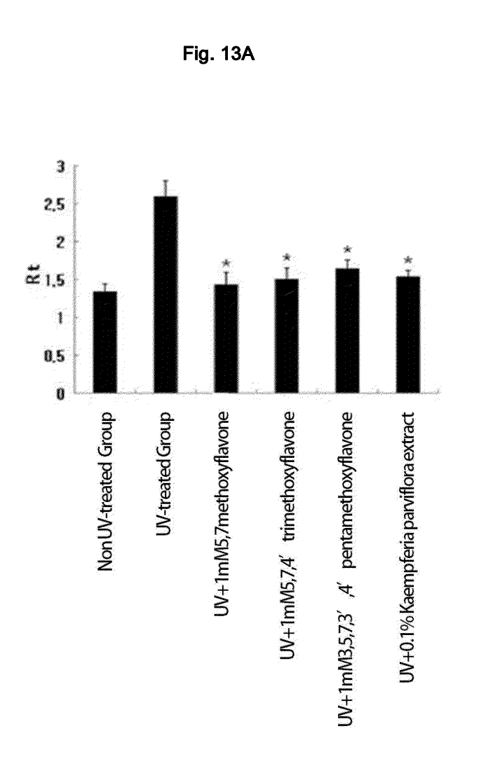

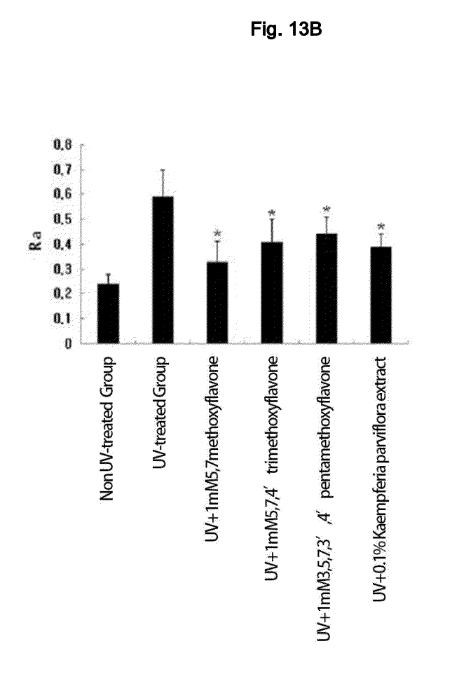

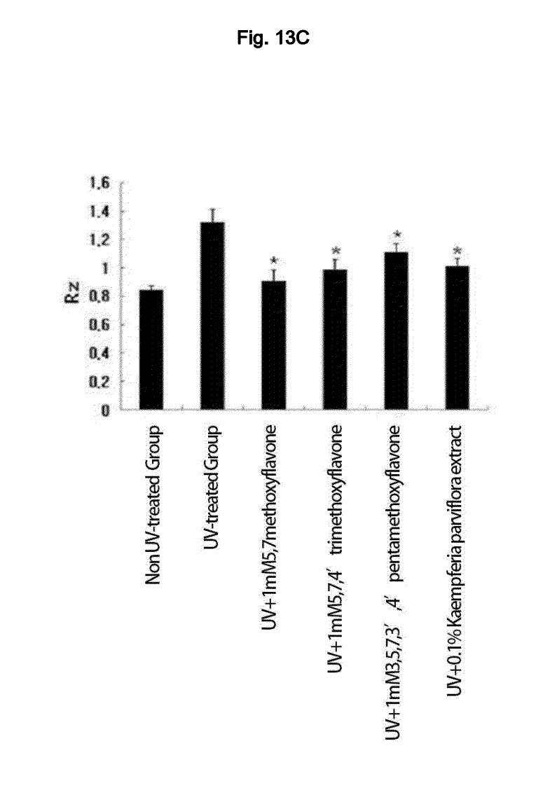

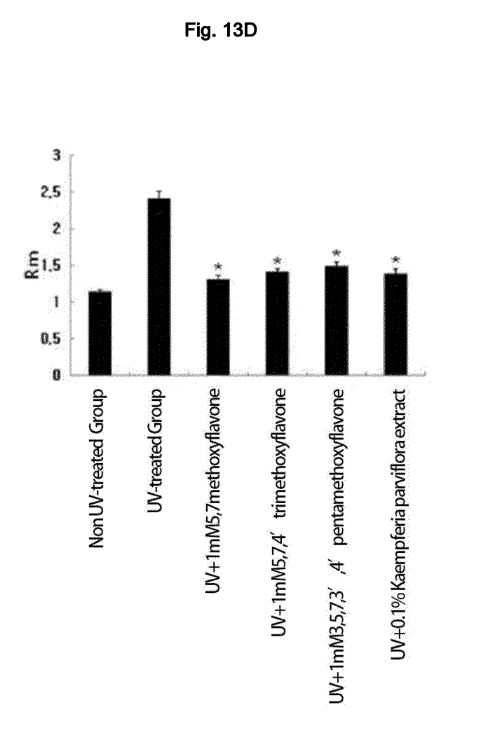

Forty eight (48) 6-week-old female hairless mice (Hos: HR-1) were accustomed for a week and randomly divided into six (6) groups, eight (8) mice per each group. The hairless mice were exposed to UV for 8 weeks. UV irradiation was carried out 3 times a week, while the amount of UV irradiation was increased from 1 MED (1 MED=50 mJ/cm²) to 4 MED which was then kept until the end of the test. Test groups were divided into a total of six groups, i.e. Non UV-treated Group, UV-treated Group, UV+5,7-dimethoxyflavone-treated Group, UV+5,7,4′-trimethoxyflavone-treated Group, UV+3,5,7,3′,4′-pentamethoxyflavone-treated Group, and UV+ethanol extract of Kaempferia parviflora-treated Group. Each sample was dissolved in a mixture solvent of ethanol and polyethylene glycol (7:3, v/v), and 50 μL of each sample was applied onto the back of the mice every day for 8 weeks. For the Non UV-treated group and the UV-treated group, 50 μL of a mixture of ethanol and polyethylene glycol (7:3, v/v) was applied. In order to test the preventative effect of wrinkle formation, skin replicas were taken using silicone polymer (SILFLO Impression Material, Flexico, England). The image files of the skin replicas were subjected to wrinkle evaluation using the computer imaging analysis system, i.e. Skin Visiometer SV 600 software (Courage+Khazaha Electronic, Kln, Germany). Rt, Rm, Rz and Ra values (Rt: the distance from the highest and lowest portions on the skin surface, Rm: the maximum Rt value among five (5) measurements, Rz: the mean Rt value of five measurements, Ra: the arithmetic mean value of surface roughness) were determined The results are shown in FIGS. 12 and 13A to 13D.

As shown in FIG. 12, 5,7-dimethoxyflavone-treated Group, 5,7,4′-trimethoxyflavone-treated Group, 3,5,7,3′,4′-pentamethoxyflavone-treated Group, and the ethanol extract of Kaempferia parviflora-treated Group showed a remarkable decrease in wrinkle formation in comparison with UV-treated Group. Also, as shown in FIGS. 13A to 13D, Rt, Rm, Rz and Ra values, which reflect the degree of wrinkle formation, were significantly reduced in 5,7-dimethoxyflavone-treated Group, 5,7,4′-trimethoxyflavone-treated Group, 3,5,7,3′,4′-pentamethoxyflavone-treated Group, and the ethanol extract of Kaempferia parviflora-treated Group (p<0.05).

Accordingly, the above test results prove that the application of 5,7-dimethoxyflavone, 5,7,4′-trimethoxyflavone, 3,5,7,3′,4′-pentamethoxyflavone, and the ethanol extract of Kaempferia parviflora onto the skin provides excellent wrinkle improvement.

### Example 7-2

### Oral Administration

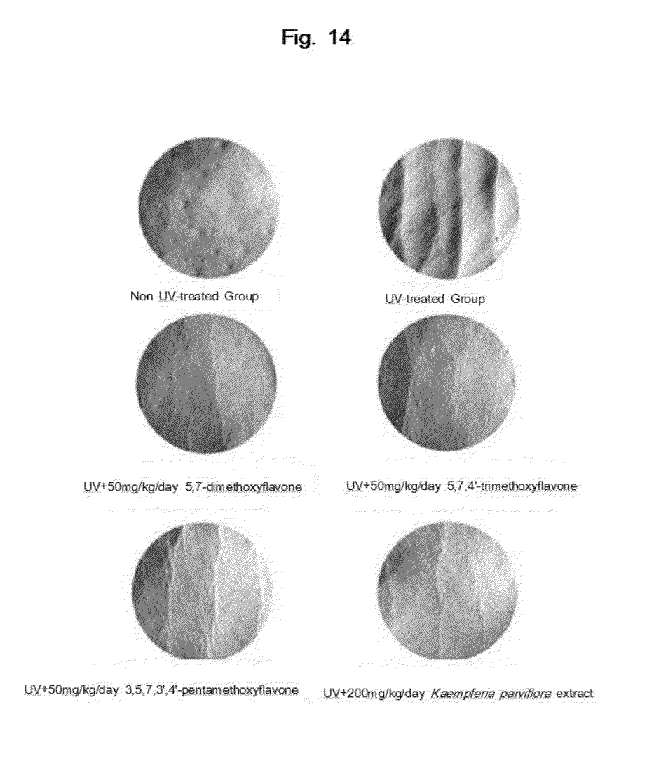

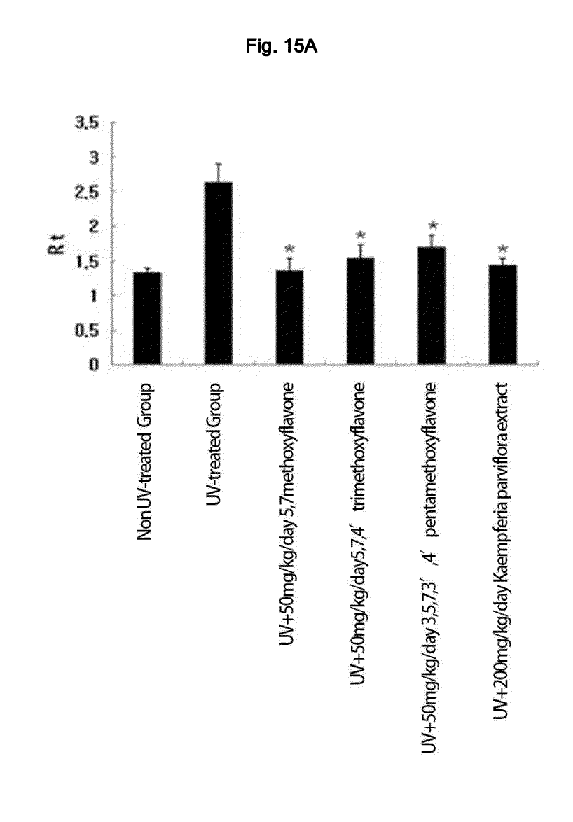

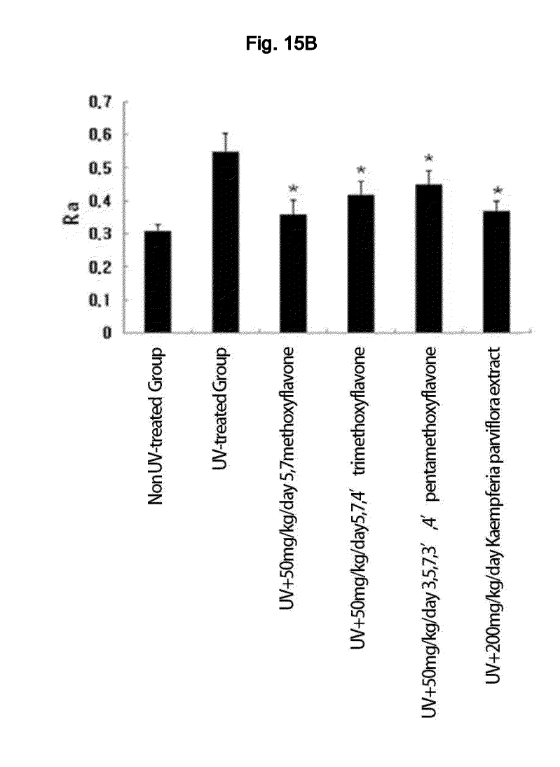

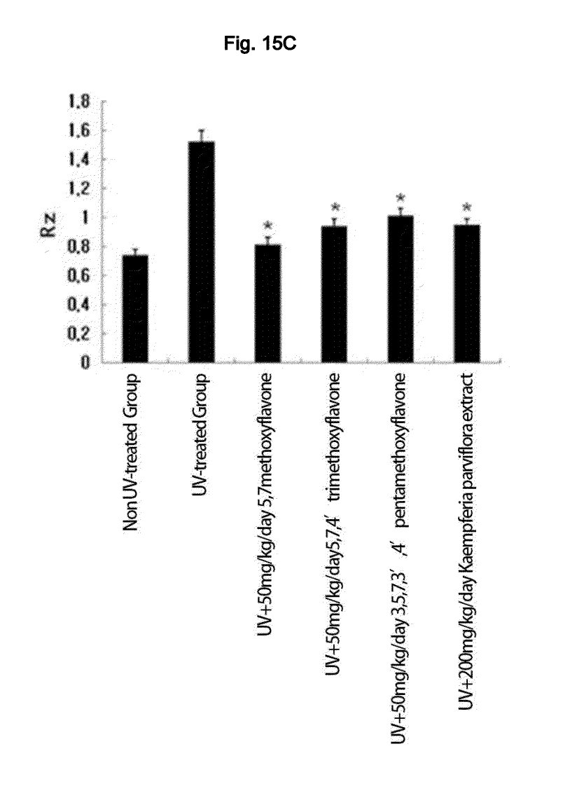

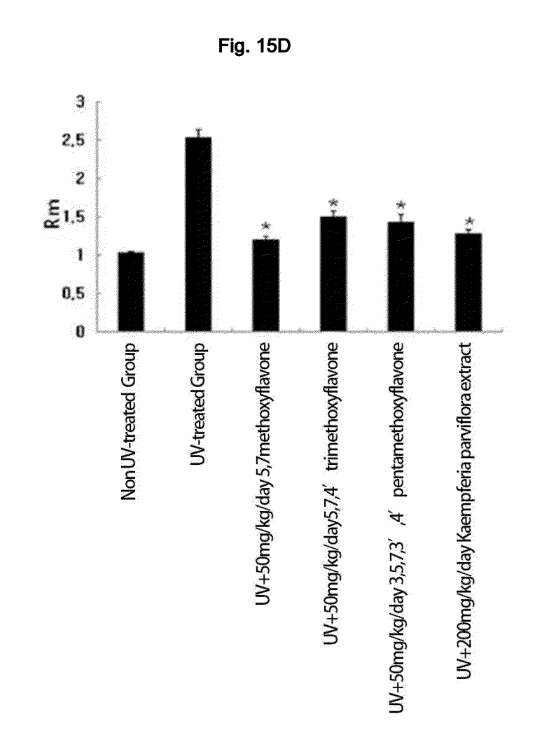

To the hairless mice exposed to UV in Example 7-1,5,7-dimethoxyflavone (50 mg/kg/day), 5,7,4′-trimethoxyflavone (50 mg/kg/day), 3,5,7,3′,4′-pentamethoxyflavone (50 mg/kg/day) and the ethanol extract of Kaempferia parviflora (200 mg/kg/day) dissolved in 0.5% carboxymethyl cellulose solution containing 5% Tween 80 were orally administered daily for 8 weeks. For the control groups (Non UV-treated Group and UV-treated Group), 0.5% carboxymethyl cellulose solution was administered. In order to test the preventative effect of wrinkle formation, skin replicas were taken using silicone polymer (SILFLO Impression Material, Flexico, England). The image files of the skin replicas were subjected to wrinkle evaluation using the computer imaging analysis system, i.e. Skin Visiometer SV 600 software (Courage+Khazaha Electronic, Kln, Germany). Rt, Rm, Rz and Ra values were determined and the result are shown in FIGS. 14 and 15A to 15D.

As shown in FIG. 14, 5,7-dimethoxyflavone-treated Group, 5,7,4′-trimethoxyflavone-treated Group, 3,5,7,3′,4′-pentamethoxyflavone-treated Group, and the ethanol extract of Kaempferia parviflora-treated Group showed a remarkable decrease in wrinkle formation compared with UV-treated Group. Also, as shown in FIGS. 15A to 15D, Rt, Rm, Rz and Ra values, which reflect the degree of wrinkle formation, were significantly reduced in 5,7-dimethoxyflavone-treated Group, 5,7,4′-trimethoxyflavone-treated Group, 3,5,7,3′,4′-pentamethoxyflavone-treated Group, and the ethanol extract of Kaempferia parviflora-treated Group (p<0.05).

Accordingly, the above test results prove that the oral administration of 5,7-dimethoxyflavone, 5,7,4′-trimethoxyflavone, 3,5,7,3′,4′-pentamethoxyflavone, and the ethanol extract of Kaempferia parviflora also greatly decreases wrinkle formation.

### Example 8

### Improvement in UV-Induced Trans-Epidermal Water Loss (TEWL)

5,7-dimethoxyflavone, 5,7,4′-trimethoxyflavone, 3,5,7,3′,4′-pentamethoxyflavone or the ethanol extract of Kaempferia parviflora as obtained in Examples 1-4 were tested to see whether they possess an improving effect on UV-induced Trans-Epidermal Water Loss (TEWL).

### Example 8-1

### Application onto the Skin

By using Tewameter (TM 300, Courage+Khazaha Electronic, Kln, Germany), Trans-Epidermal Water Loss (TEWL) was measured on the back of the mice in Example 7-1.

As shown in the following Table 2, UV-treated Group showed remarkable reduction in Trans-Epidermal Water Loss (TEWL) compared with Non UV-treated Group. Meanwhile, 5,7-dimethoxyflavone-treated Group, 5,7,4′-trimethoxyflavone-treated Group, 3,5,7,3′,4′-pentamethoxyflavone-treated Group, and the ethanol extract of Kaempferia parviflora-treated Group significantly reduced Trans-Epidermal Water Loss (TEWL) in comparison with UV-treated Group. The above test results prove that the application of 5,7-dimethoxyflavone, 5,7,4′-trimethoxyflavone, 3,5,7,3′,4′-pentamethoxyflavone, and the ethanol extract of Kaempferia parviflora onto the skin improves skin moisturization by effectively inhibiting UV-induced moisture loss from the skin.

TABLE 2
Test results for the enhancing effect on
moisture loss upon skin application

| Skin Application Test Groups | TEWL (g/m²/hr) |
|---|---|
| Non UV-treated Group | 15.5 |
| UV-treated Group | 40.0 |
| UV+ 7-dimethoxyflavone-treated Group | 19.4 |
| UV+ 5,7,4′-trimethoxyflavone-treated Group | 24.8 |
| UV+ 3,5,7,3′,4′-pentamethoxyflavone-treated | 22.0 |
| Group | |
| UV+ the ethanol extract of Kaempferia | 25.8 |
| parviflora - treated Group | |

### Example 8-2

### Oral Administration

By using Tewameter (TM 300, Courage+Khazaha Electronic, Kln, Germany), Trans-Epidermal Water Loss (TEWL) was measured on the back of the mice in Example 7-2.

As shown in the following Table 3, UV-treated Group showed remarkable increase in Trans-Epidermal Water Loss (TEWL) compared with Non UV-treated Group. Meanwhile, 5,7-dimethoxyflavone-treated Group, 5,7,4′-trimethoxyflavone-treated Group, 3,5,7,3′,4′-pentamethoxyflavone-treated Group, and the ethanol extract of Kaempferia parviflora-treated Group significantly reduced Trans-Epidermal Water Loss (TEWL) in comparison with UV-treated Group. The above test results prove that the oral administration of 5,7-dimethoxyflavone, 5,7,4′-trimethoxyflavone, 3,5,7,3′,4′-pentamethoxyflavone, and the ethanol extract of Kaempferia parviflora improves skin moisturization by effectively inhibiting UV-induced moisture loss from the skin.

TABLE 3
Test results for the enhancing effect on
moisture loss in the skin upon oral administration

| Skin Application Test Groups | TEWL (g/m²/hr) |
|---|---|
| Non UV-treated Group | 9.4 |
| UV-treated Group | 37.8 |
| UV+ 7-dimethoxyflavone-treated Group | 16.4 |
| UV+ 5,7,4′-trimethoxyflavone-treated Group | 19.6 |
| UV+ 3,5,7,3′,4′-pentamethoxyflavone-treated | 20.3 |
| Group | |
| UV+ the ethanol extract of Kaempferia | 19.4 |
| parviflora - treated Group | |

### Example 9

### Improvement in Skin Elasticity

5,7-dimethoxyflavone, 5,7,4′-trimethoxyflavone, 3,5,7,3′,4′-pentamethoxyflavone or the ethanol extract of Kaempferia parviflora as obtained in Examples 1-4 were tested to see whether they possess an improving effect on skin elasticity.

### Example 9-1

### Application onto the Skin

By using Cutometer (MPA 580, Courage+Khazaha Electronic, Germany), skin elasticity was measured on the back of the mice in the above Example 7-1 and compared with Non UV-treated Group.

As shown in the following Table 4, UV-treated Group showed remarkable reduction in skin elasticity compared with Non UV-treated Group. On the contrary, 5,7-dimethoxyflavone-treated Group, 5,7,4′-trimethoxyflavone-treated Group, 3,5,7,3′,4′-pentamethoxyflavone-treated Group, and the ethanol extract of Kaempferia parviflora-treated Group showed a remarkable improvement in skin elasticity in comparison with UV-treated Group. The above test results prove that the application of 5,7-dimethoxyflavone, 5,7,4′-trimethoxyflavone, 3,5,7,3′,4′-pentamethoxyflavone, and the ethanol extract of Kaempferia parviflora onto the skin improves skin elasticity.

TABLE 4
Test results for the enhancing effect on
skin elasticity upon skin application

| | Degree of |
|---|---|
| Skin Application Test Groups | skin elasticity (%) |
| Non UV-treated Group | 100 |
| UV-treated Group | 49.7 |
| UV+ 7-dimethoxyflavone-treated Group | 83.5 |
| UV+ 5,7,4′-trimethoxyflavone-treated Group | 76.9 |
| UV+ 3,5,7,3′,4′-pentamethoxyflavone-treated | 72.3 |
| Group | |
| UV+ the ethanol extract of Kaempferia | 80.7 |
| parviflora - treated Group | |

### Example 9-2

### Oral Administration

By using Cutometer (MPA 580, Courage+Khazaha Electronic, Germany), skin elasticity was measured on the back of the mice in Example 7-2 and compared with Non UV-treated Group.

As shown in the following Table 5, UV-treated Group showed remarkable reduction in skin elasticity compared with Non UV-treated Group. On the contrary, 5,7-dimethoxyflavone-treated Group, 5,7,4′-trimethoxyflavone-treated Group, 3,5,7,3′,4′-pentamethoxyflavone-treated Group, and the ethanol extract of Kaempferia parviflora-treated Group showed a remarkable improvement in skin elasticity in comparison with UV-treated Group. The above test results prove that the application of 5,7-dimethoxyflavone, 5,7,4′-trimethoxyflavone, 3,5,7,3′,4′-pentamethoxyflavone, and the ethanol extract of Kaempferia parviflora on the skin improves skin elasticity.

TABLE 5
Test result for the enhancing effect on
skin elasticity upon oral administration

| | Degree of |
|---|---|
| Skin Application Test Groups | skin elasticity (%) |
| Non UV-treated Group | 100 |
| UV-treated Group | 47.2 |
| UV+ 7-dimethoxyflavone-treated Group | 77.3 |
| UV+ 5,7,4′-trimethoxyflavone-treated Group | 85.6 |
| UV+ 3,5,7,3′,4′-pentamethoxyflavone-treated | 81.7 |
| Group | |
| UV+ the ethanol extract of Kaempferia | 80.5 |
| parviflora - treated Group | |

### Formulation Example 1

### Cosmetics

<1-1˜1-4> Nourishing Lotion (Milk Lotion)

Nourishing lotion was prepared according to a method commonly used in the related art by using the ethanol extract of Kaempferia parviflora or any one of 5,7-dimethoxyflavone, 5,7,4′-trimethoxyflavone and 3,5,7,3′,4′-pentamethoxyflavone of Examples 1-4, while following the chemical composition as described in Table 6 below:

TABLE 6
Nourishing Lotion (Milk Lotion)

| Ingredients | Formulation | Formulation | Formulation | Formulation |
|---|---|---|---|---|
| (wt %) | Example 1-1 | Example 1-2 | Example 1-3 | Example 1-4 |
| 5,7-dimeth- | 2.0 | — | — | — |
| oxyflavone | | | | |
| 5,7,4′-trimeth- | — | 2.0 | — | — |
| oxyflavone | | | | |
| 3,5,7,3′,4′- | — | — | 2.0 | — |
| pentameth- | | | | |
| oxyflavone | | | | |
| Extract of | — | — | — | 2.0 |
| Kaempferia | | | | |
| parviflora | | | | |
| Squalene | 5.0 | 5.0 | 5.0 | 5.0 |
| Beewax | 4.0 | 4.0 | 4.0 | 4.0 |
| Polysorbate 60 | 1.5 | 1.5 | 1.5 | 1.5 |
| Sorbitan | 1.5 | 1.5 | 1.5 | 1.5 |
| sesquioleate | | | | |
| Liquid paraffin | 0.5 | 0.5 | 0.5 | 0.5 |
| Caprylic/ | 5.0 | 5.0 | 5.0 | 5.0 |
| caprictri- | | | | |
| glyceride | | | | |
| Glycerine | 3.0 | 3.0 | 3.0 | 3.0 |
| Butylene glycol | 3.0 | 3.0 | 3.0 | 3.0 |
| Propylene | 3.0 | 3.0 | 3.0 | 3.0 |
| glycol | | | | |
| Carboxyvinyl | 0.1 | 0.1 | 0.1 | 0.1 |
| polymer | | | | |
| Triethanol- | 0.2 | 0.2 | 0.2 | 0.2 |
| amine | | | | |
| Preservative, | adequate | adequate | adequate | adequate |
| Pigment, | | | | |
| Flavoring agent | | | | |
| Purified water | to 100 | to 100 | to 100 | to 100 |

1-5˜1-8> Softening Lotion (Skin Lotion)

Softening lotion was prepared according to a method commonly used in the related art by using the ethanol extract of Kaempferia parviflora or any one of 5,7-dimethoxyflavone, 5,7,4′-trimethoxyflavone and 3,5,7,3′,4′-pentamethoxyflavone of Examples 1-4, while following the chemical composition as described in Table 7 below:

TABLE 7
Softening Lotion (Skin Lotion)

| Ingredients | Formulation | Formulation | Formulation | Formulation |
|---|---|---|---|---|
| (wt %) | Example 1-5 | Example 1-6 | Example 1-7 | Example 1-8 |
| 5,7-dimethoxy- | 2.0 | — | — | — |
| flavone | | | | |
| 5,7,4′-trimeth- | — | 2.0 | — | — |
| oxyflavone | | | | |
| 3,5,7,3′,4′- | — | — | 2.0 | — |
| pentamethoxy- | | | | |
| flavone | | | | |
| Extract of | — | — | — | 2.0 |
| Kaempferia | | | | |
| parviflora | | | | |
| Glycerine | 3.0 | 3.0 | 3.0 | 3.0 |
| Butylene glycol | 2.0 | 2.0 | 2.0 | 2.0 |
| Propylene | 2.0 | 2.0 | 2.0 | 2.0 |
| glycol | | | | |
| Carboxyvinyl | 0.1 | 0.1 | 0.1 | 0.1 |
| polymer | | | | |
| PEG 12 | 0.2 | 0.2 | 0.2 | 0.2 |
| nonylphenyl | | | | |
| ether | | | | |
| Polysorbate 60 | 0.4 | 0.4 | 0.4 | 0.4 |
| Ethanol | 10.0 | 10.0 | 10.0 | 10.0 |
| Triethanol- | 0.1 | 0.1 | 0.1 | 0.1 |
| amine | | | | |
| Preservative, | adequate | adequate | adequate | adequate |
| Pigment, | | | | |
| Flavoring agent | | | | |
| Purified water | to 100 | to 100 | to 100 | to 100 |

1-9˜1-12> Nourishing Cream

Nourishing cream was prepared according to a method commonly used in the related art by using the ethanol extract of Kaempferia parviflora or any one of 5,7-dimethoxyflavone, 5,7,4′-trimethoxyflavone and 3,5,7,3′,4′-pentamethoxyflavone of Examples 1-4, while following the chemical composition as described in Table 8 below:

TABLE 8
Nourishing cream

| | | Formulation | Formulation | Formulation |
|---|---|---|---|---|
| Ingredients | Formulation | Example | Example | Example |
| (wt %) | Example 1-9 | 1-10 | 1-11 | 1-12 |
| 5,7-dimethoxy- | 2.0 | — | — | — |
| flavone | | | | |
| 5,7,4′-trimeth- | — | 2.0 | — | — |
| oxyflavone | | | | |
| 3,5,7,3′,4′- | — | — | 2.0 | — |
| pentamethoxy- | | | | |
| flavone | | | | |
| Extract of | — | — | — | 2.0 |
| Kaempferia | | | | |
| parviflora | | | | |
| Polysorbate 60 | 1.5 | 1.5 | 1.5 | 1.5 |
| Sorbitan | 0.5 | 0.5 | 0.5 | 0.5 |
| sesquioleate | | | | |
| PEG 60 | 2.0 | 2.0 | 2.0 | 2.0 |
| hydrogenated | | | | |
| castor oil | | | | |
| Liquid paraffin | 10 | 10 | 10 | 10 |
| Squalene | 5.0 | 5.0 | 5.0 | 5.0 |
| Caprylic/ | 5.0 | 5.0 | 5.0 | 5.0 |
| caprictri- | | | | |
| glyceride | | | | |
| Glycerine | 5.0 | 5.0 | 5.0 | 5.0 |
| Butylene glycol | 3.0 | 3.0 | 3.0 | 3.0 |
| Propylene | 3.0 | 3.0 | 3.0 | 3.0 |
| glycol | | | | |
| Triethanol- | 0.2 | 0.2 | 0.2 | 0.2 |
| amine | | | | |
| Preservative | adequate | adequate | adequate | adequate |
| Pigment | adequate | adequate | adequate | adequate |
| Flavoring agent | adequate | adequate | adequate | adequate |
| Purified water | to 100 | to 100 | to 100 | to 100 |

<1-13˜1-16> Massage Cream

Massage cream was prepared according to a method commonly used in the related art by using the ethanol extract of Kaempferia parviflora or any one of 5,7-dimethoxyflavone, 5,7,4′-trimethoxyflavone and 3,5,7,3′,4′-pentamethoxyflavone of Examples 1-4, while following the chemical composition as described in Table 9 below:

TABLE 9
Massage cream

| | Formulation | Formulation | Formulation | Formulation |
|---|---|---|---|---|
| Ingredients | Example | Example | Example | Example |
| (wt %) | 1-13 | 1-14 | 1-15 | 1-16 |
| 5,7-dimethoxy- | 1.0 | — | — | — |
| flavone | | | | |
| 5,7,4′-trimeth- | — | 1.0 | — | — |
| oxyflavone | | | | |
| 3,5,7,3′,4′- | — | — | 1.0 | — |
| pentamethoxy- | | | | |
| flavone | | | | |
| Extract of | — | — | — | 1.0 |
| Kaempferia | | | | |
| parviflora | | | | |
| Beewax | 10.0 | 10.0 | 10.0 | 10.0 |
| Polysorbate 60 | 1.5 | 1.5 | 1.5 | 1.5 |
| PEG 60 | 2.0 | 2.0 | 2.0 | 2.0 |
| hydrogenated | | | | |
| castor oil | | | | |
| Sorbitan | 0.8 | 0.8 | 0.8 | 0.8 |
| sesquioleate | | | | |
| Liquid paraffin | 40.0 | 40.0 | 40.0 | 40.0 |
| squalene | 5.0 | 5.0 | 5.0 | 5.0 |
| Caprylic/capric | 4.0 | 4.0 | 4.0 | 4.0 |
| triglyceride | | | | |
| Glycerine | 5.0 | 5.0 | 5.0 | 5.0 |
| Butylene glycol | 3.0 | 3.0 | 3.0 | 3.0 |
| Propylene | 3.0 | 3.0 | 3.0 | 3.0 |
| glycol | | | | |
| Triethanol- | 0.2 | 0.2 | 0.2 | 0.2 |
| amine | | | | |
| Preservative, | Adequate | adequate | adequate | adequate |
| Pigment, | | | | |
| Flavoring agent | | | | |
| Purified water | to 100 | to 100 | to 100 | to 100 |

<1-17˜1-20> Pack

Pack was prepared according to a method commonly used in the related art by using the ethanol extract of Kaempferia parviflora or any one of 5,7-dimethoxyflavone, 5,7,4′-trimethoxyflavone and 3,5,7,3′,4′-pentamethoxyflavone of Examples 1-4, while following the chemical composition as described in Table 10 below:

TABLE 10
Pack

| | Formulation | Formulation | Formulation | Formulation |
|---|---|---|---|---|
| Ingredients | Example | Example | Example | Example |
| (wt %) | 1-17 | 1-18 | 1-19 | 1-20 |
| 5,7-dimethoxy- | 1.0 | — | — | — |
| flavone | | | | |
| 5,7,4′-trimeth- | — | 1.0 | — | — |
| oxyflavone | | | | |
| 3,5,7,3′,4′- | — | — | 1.0 | — |
| pentamethoxy- | | | | |
| flavone | | | | |
| Extract of | — | — | — | 1.0 |
| Kaempferia | | | | |
| parviflora | | | | |
| Polyvinyl | 13.0 | 13.0 | 13.0 | 13.0 |
| alcohol | | | | |
| Sodium | 0.2 | 0.2 | 0.2 | 0.2 |
| carboxymethyl | | | | |
| cellulose | | | | |
| Glycerine | 5.0 | 5.0 | 5.0 | 5.0 |
| Allantoin | 0.1 | 0.1 | 0.1 | 0.1 |
| Ethanol | 6.0 | 6.0 | 6.0 | 6.0 |
| PEG 12 | 0.3 | 0.3 | 0.3 | 0.3 |
| Nonylpheny- | | | | |
| lether | | | | |
| Polysorbate 60 | 0.3 | 0.3 | 0.3 | 0.3 |
| Preservative, | Adequate | adequate | adequate | adequate |
| Pigment, | | | | |
| Flavoring agent | | | | |
| Purified water | to 100 | to 100 | to 100 | to 100 |

1-21˜1-24> Gel

Gel was prepared according to a method commonly used in the related art by using the ethanol extract of Kaempferia parviflora or any one of 5,7-dimethoxyflavone, 5,7,4′-trimethoxyflavone and 3,5,7,3′,4′-pentamethoxyflavone of Examples 1-4, while following the chemical composition as described in Table 11 below:

TABLE 11
Gel

| | Formulation | Formulation | Formulation | Formulation |
|---|---|---|---|---|
| Ingredients | Example | Example | Example | Example |
| (wt %) | 1-21 | 1-22 | 1-23 | 1-24 |
| 5,7-dimethoxy- | 0.5 | — | — | — |
| flavone | | | | |
| 5,7,4′-trimeth- | — | 0.5 | — | — |
| oxyflavone | | | | |
| 3,5,7,3′,4′- | — | — | 0.5 | — |
| pentamethoxy- | | | | |
| flavone | | | | |
| Extract of | — | — | — | 0.5 |
| Kaempferia | | | | |
| parviflora | | | | |
| Ethylene- | 0.05 | 0.05 | 0.05 | 0.05 |
| diamine | | | | |
| sodium acetate | | | | |
| Glycerine | 5.0 | 5.0 | 5.0 | 5.0 |
| Carboxyvinyl | 0.3 | 0.3 | 0.3 | 0.3 |
| polymer | | | | |
| Ethanol | 5.0 | 5.0 | 5.0 | 5.0 |
| PEG 60 | 0.5 | 0.5 | 0.5 | 0.5 |
| hydrogenated | | | | |
| castor oil | | | | |
| Triethanol- | 0.3 | 0.3 | 0.3 | 0.3 |
| amine | | | | |
| Preservative, | Adequate | adequate | adequate | adequate |
| Pigment, | | | | |
| Flavoring agent | | | | |
| Purified water | to 100 | to 100 | to 100 | to 100 |

### Formulation Example 2

### Health Food

<2-1> Preparation of Health Food

1,000 mg of the ethanol extract of Kaempferia parviflora or any one of 5,7-dimethoxyflavone, 5,7,4′-trimethoxyflavone and 3,5,7,3′,4′-pentamethoxyflavone of Examples 1-4 may be mixed with 70 μg of vitamin A acetate, 1.0 mg of vitamin E, 0.13 mg of vitamin B1, 0.15 mg of vitamin B2, 0.5 mg of vitamin B6, 0.2 μg of vitamin B12, 10 mg of vitamin C, 10 μg of biotin, 1.7 mg of nicotinic acid amide, 50 μg of folic acid, 0.5 mg of calcium pantothenate, 1.75 mg of ferrous sulfate, 0.82 mg of zinc oxide, 25.3 mg of magnesium carbonate, 15 mg of monobasic potassium phosphate, 55 mg of dibasic calcium phosphate, 90 mg of potassium citrate, 100 mg of calcium carbonate and 24.8 mg of magnesium chloride. The mixing ratio of the said ingredients may be changed differently. The mixture may be prepared into granules according to a method commonly used in the related art, and may then be used for the preparation of a health food composition according to a method commonly used in the related art.

<2-2> Preparation of Health Drink

1,000 mg of the ethanol extract of Kaempferia parviflora or any one of 5,7-dimethoxyflavone, 5,7,4′-trimethoxyflavone and 3,5,7,3′,4′-pentamethoxyflavone of Examples 1-4 may be mixed with 1,000 mg of citric acid, 100 g of oligosaccharide, 2 g of plum extract and 1 g of taurine according to a method commonly used in the related art. Purified water may then be added to produce a total volume of 900 mL. After heating at 85° C. for about 1 hour while stirring, the resultant solution may be filtered and collected in a sterilized 2 L container. After sealing and sterilization, it may be kept under refrigeration for the preparation of a health drink composition.

<2-3> Chewing Gum

0.1 wt % of the ethanol extract of Kaempferia parviflora or any one of 5,7-dimethoxyflavone, 5,7,4′-trimethoxyflavone and 3,5,7,3′,4′-pentamethoxyflavone of Examples 1-4 was mixed with 20 wt % of gum base, 76.9 wt % of sugar, 1 wt % of flavoring agent and 2 wt % of water according to a method commonly used in the related art to prepare chewing gum.

<2-4> Candy

0.1 wt % of the ethanol extract of Kaempferia parviflora or any one of 5,7-dimethoxyflavone, 5,7,4′-trimethoxyflavone and 3,5,7,3′,4′-pentamethoxyflavone of Examples 1-4 was mixed with 60 wt % of sugar, 39.8 wt % of starch syrup and 0.1 wt % of flavoring agent according to a method commonly used in the related art to prepare candy.

<2-5> Biscuit

1 wt % of the ethanol extract of Kaempferia parviflora or any one of 5,7-dimethoxyflavone, 5,7,4′-trimethoxyflavone and 3,5,7,3′,4′-pentamethoxyflavone of Examples 1-4 was mixed with 25.59 wt % of hard wheat flour, 22.22 wt % of medium wheat flour, 4.80 wt % of refined sugar, 0.73 wt % of table salt, 0.78 wt % of glucose, 11.78 wt % of palm shortening, 1.54 wt % of ammonium, 0.17 wt % of baking soda, 0.16 wt % of sodium bisulfite, 1.45 wt % of rice flour, 0.0001 wt % of vitamin B, 0.0001 wt % of vitamin B, 0.04 wt % of milk flavor, 20.6998 wt % of water, 1.16 wt % of whole milk powder, 0.29 wt % of milk replacer, 0.03 wt % of monobasic calcium phosphate, 0.29 wt % of scattering salt and 7.27 wt % of spray-milk according to a method commonly used in the related art to prepare biscuit.

### Formulation Example 3

### Drugs

<3-1> Powder

50 mg of the ethanol extract of Kaempferia parviflora or any one of 5,7-dimethoxyflavone, 5,7,4′-trimethoxyflavone and 3,5,7,3′,4′-pentamethoxyflavone of Examples 1-4 was mixed with 2 g of crystalline cellulose and put in an air-tight pouch according to a method commonly used in the related art to prepare powder.

<3-2> Tablet

50 mg of the ethanol extract of Kaempferia parviflora or any one of 5,7-dimethoxyflavone, 5,7,4′-trimethoxyflavone and 3,5,7,3′,4′-pentamethoxyflavone of Examples 1-4 was mixed with 400 mg of crystalline cellulose and 5 mg of magnesium stearate, then followed by a method commonly used in the related art to prepare tablet.

<3-3> Capsule

30 mg of the ethanol extract of Kaempferia parviflora or any one of 5,7-dimethoxyflavone, 5,7,4′-trimethoxyflavone and 3,5,7,3′,4′-pentamethoxyflavone of Examples 1-4 was mixed with 100 mg of whey protein, 400 mg of crystalline cellulose and 6 mg of magnesium stearate, then followed by a method commonly used in the related art to prepare capsule.

<3-4> Injection Solution

According to a method commonly used in the related art, active ingredients were dissolved in distilled water for injection, while pH was adjusted to about 7.5. Then, 100 mg of the ethanol extract of Kaempferia parviflora or any one of 5,7-dimethoxyflavone, 5,7,4′-trimethoxyflavone and 3,5,7,3′,4′-pentamethoxyflavone of Examples 1-4 was mixed with distilled water for injection and pH regulator, then put into in a 2 ml ampule and sterilized to prepare injection solution.

### INDUSTRIAL APPLICABILITY

The present invention provides a cosmetic, food and pharmaceutical composition for wrinkle improvement, anti-aging and skin elasticity enhancement or a cosmetic, food and pharmaceutical composition for skin moisturization, which comprises a flavone-based compound or a Kaempferia parviflora extract comprising the same as an active ingredient. The compositions according to the present invention are highly useful in terms of industrial applicability since they are excellent in inhibiting the activity of Collagenase-1 and promoting the synthesis of collagen, resulting in being effective for wrinkle improvement, anti-aging and skin elasticity enhancement, while being significantly effective in reducing moisture loss from the skin and improving skin moisturization.

1. A cosmetic, food, or pharmaceutical composition for wrinkle improvement, anti-aging and skin elasticity enhancement or a cosmetic or food composition for skin moisturization comprising one or more flavone-based compound represented by Chemical Formulas 1 to 3 or its salt thereof as an active ingredient:

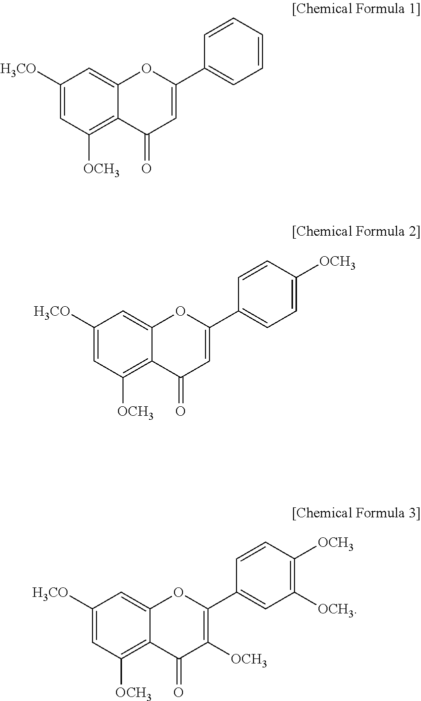 [SMILES: COc1cc(OC)c2c(=O)cc(-c3ccccc3)oc2c1 ; COc1ccc(-c2cc(=O)c3c(OC)cc(OC)cc3o2)cc1 ; COc1cc(OC)c2c(=O)c(OC)c(-c3ccc(OC)c(OC)c3)oc2c1]

2-5. (canceled)

6. A cosmetic, food, or pharmaceutical composition for wrinkle improvement, anti-aging and skin elasticity enhancement or a cosmetic or food composition for skin moisturization comprising a Kaempferia parviflora extract as an active ingredient.

7-10. (canceled)

11. The composition according to claim 6, wherein the Kaempferia parviflora extract is extracted with one or more solvent selected from the group consisting of water, C₁-C₆ organic solvent, and subcritical or supercritical fluid.

12. The composition of claim 11, wherein the C₁-C₆ organic solvent is selected from the group consisting of C₁-C₆ alcohol, acetone, ether, benzene, chloroform, ethyl acetate, methylene chloride, hexane, cyclohexane, and petroleum ether.

13. (canceled)

14. A method for wrinkle improvement, anti-aging and skin elasticity enhancement or skin moisturization comprising administering or applying to a subject in need thereof an effective amount of one or more flavone-based compound represented by Chemical Formulas 1 to 3 or its pharmaceutically acceptable salt thereof.

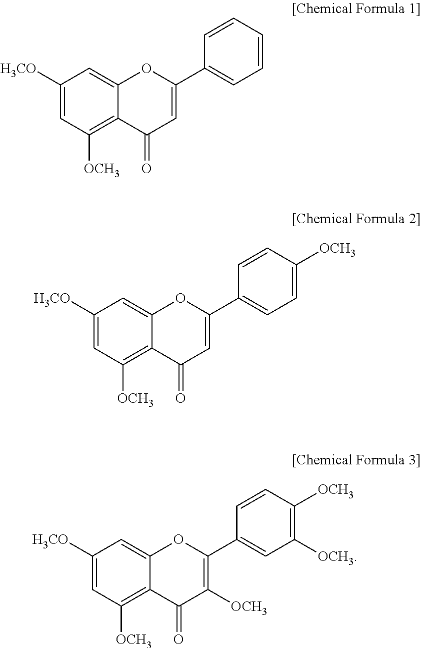 [SMILES: COc1cc(OC)c2c(=O)cc(-c3ccccc3)oc2c1 ; COc1ccc(-c2cc(=O)c3c(OC)cc(OC)cc3o2)cc1 ; COc1cc(OC)c2c(=O)c(OC)c(-c3ccc(OC)c(OC)c3)oc2c1]

15-17. (canceled)

18. A method for wrinkle improvement, anti-aging and skin elasticity enhancement or for skin moisturization comprising administering or applying to a subject in need thereof an effective amount of a composition of claims 6.

19-20. (canceled)
